(19) United States
(12) Patent Application Publication
(10) Pub. No.: US 2013/0196915 A1
(43) Pub. Date: Aug. 1, 2013

(54) AFFINITY HYDROGELS FOR CONTROLLED PROTEIN RELEASE

(71) Applicants: Yong Wang, Willington, CT (US); Boonchoy Soontornworajit, Storrs, CT (US); Niancao Chen, Storrs, CT (US)

(72) Inventors: Yong Wang, Willington, CT (US); Boonchoy Soontornworajit, Storrs, CT (US); Niancao Chen, Storrs, CT (US)

(21) Appl. No.: 13/522,837

(22) PCT Filed: Jan. 21, 2011
(86) PCT No.: PCT/US11/22128

§ 371 Date: Mar. 7, 2013

Related U.S. Application Data
(60) Provisional application No. 61/336,491, filed on Jan. 23, 2010.

(51) Int. Cl. A61K 47/48

(52) U.S. Cl. CPC A61K 47/48092
USPC 514/11.4; 514/20.9; 514/11.3

(57) ABSTRACT

The present invention relates to novel porous matrix composites and formulations for controlled protein delivery and the uses therefor. The present invention also provides methods of synthesizing such protein delivery systems. The composites comprise affinity sites embedded in the matrix where the affinity sites are functionalized with nucleic acid aptamers having high affinity for proteins to be released. The aptamers function as binding affinity sites for the proteins to be released. In certain embodiments, release rates are controlled by tuning the binding affinity of the nucleic acid aptamers to the proteins at a desired level. In yet other embodiments, complementary oligonucleotides that hybridize with the aptamers are employed to trigger accelerated release of the proteins when desired. Various in situ injectable hydro gels functionalized with aptamers are provided for treating a condition and disease in a subject in need of a therapeutic protein.

### RELATED APPLICATION(S)

This application claims the benefit of U.S. Provisional Application No. 61/336,491, filed on Jan. 23, 2010.

The entire teachings of the above application are incorporated herein by reference.

### GOVERNMENT SUPPORT

The present invention was developed in part with funding from the National Science Foundation under Grant # DMR 0705716. The United States Government has certain rights in this invention.

### BACKGROUND OF THE INVENTION

Proteins are potent in controlling cell behavior and carry a great potential for disease treatment as therapeutic agents. However, traditional drug delivery methods that rely on subcutaneous, intramuscular, or intravenous injections of liquid formulations have been found to be unsuitable for protein delivery and release because many proteins have short in vivo half-lives and are easily degraded by enzymes before reaching target sites with a sufficient concentration. For example, the half-lives of platelet-derived growth factor (PDGF), basic fibroblast growth factor (bFGF), and vascular endothelial growth factor (VEGF) in blood circulation are only 2, 3, and 5 minutes, respectively. As a result, multiple injections and substantial increases of doses are often necessary to produce therapeutic effects. They not only raise the treatment costs, but also lead to a wide distribution of protein drugs in non-target tissues and generate severe systemic side-effects.

Polymeric hydrogel systems as protein vehicles have been proposed as an alternative approach for protein delivery. Conventionally, protein drugs have been incorporated into hydrogels for their delivery by directly loading them into the hydrogel matrix. Protein release can be controlled either through passive diffusion or by environmental stimuli (e.g., enzymatic digestion of hydrogel networks and mechanical force). However, hydrogels have high permeability, which often leads to fast substance release. Without specific modification of hydrogels, protein drugs exhibit a rapid burst release during the initial phase of hydrogel swelling and then an extended release of the proteins left in the hydrogel network. The problem with the uncontrolled rapid release has not only decreased the biological efficacy of the released proteins, but also caused severe side-effects in vivo.

Further, unlike conventional low molecular weight drugs, proteins are large molecules possessing secondary, tertiary, and even quaternary structure with labile bonds and side chains. Structural disruption or chain modification due to the harsh conditions for producing synthetic hydrogels can result in a significant loss of protein bioactivity. The use of organic solvents, the dispersion under mechanical force, and the conjugation with covalent bonds often inactivate or denature a significant amount of proteins during the preparation processes of polymeric hydrogels. Moreover, the recent studies have shown that there are high levels of immunogenicity and cytotoxicity in conventional polymeric systems used for protein delivery (e.g., heparin or Ni²⁺ containing polymeric systems). Moreover, because the methods for the preparation of the conventional protein release systems often involve complicated procedures which significantly reduces the consistency and reproducibility.

Therefore, there is a need in the art to provide controllable peptide or protein release compositions that are easy-to-make, economical, safe and reliable and that release the peptides or proteins at a desired rate in a subject.

### SUMMARY OF THE INVENTION

The present invention provides composition, systems and methods for controlled release of peptide or proteins. In one embodiment, the present invention comprises affinity hydrogel compositions, formulations, methods and materials for peptide or protein delivery useful in a wide range of medical, pharmaceutical and agricultural applications. Thus, the present invention relates to a technology that provides easy-to-manufacture methods and reproducible compositions and formulations for peptide or protein delivery for treating a disease in a subject.

According to the present invention, nucleic acid aptamers are used to provide novel affinity sites in hydrogels or other porous matrices for entrapping peptides or proteins in these matrices because of their high binding affinity and specificity.

In one specific embodiment of the present invention, a composition for the controlled release of peptides or proteins comprises, for example, a hydrogel having a plurality of affinity sites comprising, for example, micro- or nono-particles and one or more nucleic acid aptamers attached to the affinity particles. The particles are coated, tethered, attached, or conjugated with one or more functional nucleic acid aptamers and the nucleic acid aptamers bind to one or more peptides or proteins with high affinity and specificity. The nucleic acid aptamers can be one or more types having binding affinity and specificity to one or more types of peptides or proteins. According to some embodiments of the invention, the peptides or proteins can be loaded onto the affinity sites before or after the polymerization and gelation of the hydrogel. In some instances, the peptides or proteins are saturated on the surface of the affinity particles comprising one or more types of nucleic acid aptamers before the polymerization and gelation. The peptides or proteins can be loaded onto the aptamer-functionalized affinity particles by mixing the peptide or proteins with the aptamer-functionalized affinity particles.

Nucleic acid aptamers can be selected from oligonucleotide libraries for the protein(s) of interest. The nucleic acid aptamers can be selected from oligonucleotide libraries for their affinity to the peptides or proteins whose release is to be controlled. The nucleic acid aptamers can also have high specificity and can discriminate targets on the basis of subtle structural differences. Further, the affinity of the nucleic acid aptamers to the peptides or proteins can be adjusted by modifying the sequence and the length of the aptamers, thereby controlling the release of the peptides or proteins. In a specific embodiment, the aptamers also have tunable stability in biological environments and their biodegradability can be controlled by the degree of nucleotide modification. In certain embodiments, nucleic acid aptamers have little or no immunogenicity or toxicity. In yet other embodiments, the binding functionality of nucleic acid aptamers can be inactivated on demand. In addition, the nucleic acid aptamers can be biotinylated to facilitate the binding to streptavidin-coated affinity particles that are incorporated in the hydrogel networks.

According to another embodiment of the invention, the affinity particles can be biocompatible beads of various sizes. Preferably, the beads can be microbeads or nanobeads whose sizes range, for example, from about 0.5 μm to about 500 μm in diameter. Further, the particles can be uniform polymer particles, for example, uniform polystyrene particles. In some embodiments, the particles can be conjugated, coated, tethered, or chemically bonded to the nucleic acid aptamers. In one embodiment, the particles are coated with streptavidin to facilitate the conjugation with the biotinylated nucleic acid aptamers.

According to another embodiment of the present invention, a method of controlling protein release from a hydrogel comprising a plurality of micro- or nano-particles conjugated with high affinity nucleic acid aptamers that bind to peptides or proteins whose release from the hydrogel is to be controlled is provided. The peptides or proteins are bound to the high affinity nucleic acid aptamers at predetermined affinity suitable for a desired release rate. In one embodiment, the release kinetics of the hydrogel functionalized with nucleic acid aptamers is slower as compared with that of the hydrogels that are not functionalized with the nucleic acid aptamers. The release kinetics can be tuned by modulating the binding affinity of nucleic acid aptamers selected for the protein. In yet another embodiment, complementary oligonucleotides (COs) that are designed to bind or hybridize to nucleic acid aptamers can be used as molecular triggers that further regulate, modulate or accelerate the release of peptides or proteins from nucleic acid aptamers in the hydrogel network. The nucleic acid aptamers and/or complementary oligonucleotides are optionally labeled, such as fluorescently labeled, for detection. In some embodiment, the peptides or proteins are released for a defined time period and/or at a predetermined amount.

A method for making a formulation for controlled peptide or protein release is also provided. The method comprises the steps of (a) coating or conjugating nucleic acid aptamers on the surface of micro- or nano-particles, (b) contacting the nucleic acid aptamer-coated particles with peptides or proteins having binding affinity to the nucleic acid aptamers, (c) allowing the peptides or proteins to bind to the nucleic acid aptamers, (d) transferring the aptamer-coated particles saturated with the peptides or proteins into a pre-gelation polymer solution, and (e) forming a gel. The gel can be formed under physiological conditions in which the peptides or proteins retain original function. In still another embodiment, the gel is a hydrogel which is biocompatible. In the technology of the present invention, the preparation of protein release formations is simple and reproducible. In the methods of the present invention, peptides or proteins can be simply mixed with aptamer-tethered micro-/nano-particles to facilitate binding between the aptamers and the peptides or proteins.

A method for delivering peptides or proteins to a subject in need of such peptides or proteins is also provided. The method comprises providing a formulation comprising a hydrogel having affinity sites that comprise nucleic acid aptamers having high affinity to the peptides or proteins in a physiologically effective amount to the subject in need of the peptides or proteins. The subject can be a human patient in need of the protein for treatment in, for example, short stature, Turner's syndrome or chronic renal failure. For short stature, the proteins can be human growth hormone. The subject can also be a live stock. For example, if the live stock is a dairy cow, the proteins can be bovine somatotropin.

One of ordinary skill in the art would appreciate that the present invention can be applied to situations in which it is desired to deliver peptides or proteins in a reproducible and controlled manner to treat a disease or condition or to create an artificial scaffold for inducing tissue regeneration and remodeling to recapitulate the features of extracellular matrices.

In one embodiment of the present invention, the present invention provides a formulation comprising a protein release system for mammals such as humans, apes, monkeys, cattle, horses, sheep, goats, swine, rabbits, dogs, cats, rats, mice, guinea pigs, and the like. In yet another embodiment, the formulation comprises release system for non-mammals such as reptiles, birds, fish and the like. For example, growth hormones can be released from the formulation of the present invention to treat short stature in human or for stimulating milk production in dairy cows.

In the present invention, protein release from a formulation can be decoupled from gel degradation. Thus, the release does not rely on degradation of hydrogels providing an advantage over the conventional approaches in that the mechanical properties and integrity of hydrogels is maintained during the desired time period of administration and controlled protein release.

The present invention has various advantages over conventional technologies. Nucleic acid aptamers can entrap one or multiple types of peptides or proteins in hydrogel matrix because of their high binding affinity and specificity. Complementary oligonucleotides (COs) can be designed as molecular triggers to modulate the interactions between nucleic acid aptamers and the proteins. Based on this design, the release kinetics of different peptide or protein drugs can be readily controlled. Hydrogels can be formed in a mild physiological condition and all materials used for hydrogel preparation and release control can be biocompatible. Accordingly, no toxic molecules or harsh conditions are involved during hydrogel preparation, protein loading, and protein release. Therefore, the present invention solves the problems in the conventional protein delivery systems based on hydrogels: the high permeability of matrix, the inefficiency of controlling the release of multiple proteins, and the involvement of toxic molecules and/or harsh conditions during the preparation of protein delivery systems.

### BRIEF DESCRIPTION OF THE DRAWINGS

FIG. 1 depicts an exemplary structure and composition of a hydrogel (A), and two different mechanisms for protein release from that composition (B) and (C). In (B), protein release is controlled through passive dissociation. In (C), protein release is controlled through complementary oligonucleotide-mediated intermolecular hybridization.

FIG. 2(A) depicts the secondary structures of higher-affinity aptamer and FIG. 2(B) depicts the secondary structure of lower-affinity aptamer. FIG. 2(C) depicts surface plasmon resonance (SPR) binding profiles (blue line: higher-affinity aptamer (K_{D}=25 nM); red line: lower-affinity aptamer (K_{D}=220 nM)).

FIG. 3 depicts porous matrix synthesis and characterization. FIG. 3(A) depicts a schematic representation of polyacrylamide hydrogel synthesis for sustained release. FIG. 3(B) depicts hydrogel staining and imaging, in which hydrogels were stained with ethidium bromide after gel electrophoresis: (1) native hydrogel; (2) hydrogel physically mixed with aptamer (no chemical conjugation); (3) hydrogel functionalized with lower-affinity aptamer; and (4) hydrogel functionalized with higher-affinity aptamer. FIG. 3(C) depicts the measurement of storage (unfilled markers) and loss (filled markers) modulus. (⋄, ♦) Native hydrogel; (□, ▪) hydrogel functionalized with lower-affinity aptamer; and (Δ, ▴) hydrogel functionalized with lower-affinity aptamer.

FIG. 4 depicts cumulative release of PDGF-BB from hydrogels. (♦) Native hydrogel; (▪) control aptamer-functionalized hydrogel; (▴) anti-PDGF-BB aptamer-functionalized hydrogel. Symbols and curves represent experimental results and theoretical analysis, respectively.

FIG. 5 depicts the effect of complementary oligonucleotides (COs) on the dissociation of the 36-nt aptamer from PDGF-BB. FIG. 5(A) depicts the aptamer structure and hybridization regions of four COs. FIG. 5(B) shows a gel electrophoresis image. The aptamer was alone (apt) or mixed with CO-1 (1), CO-2 (2), CO-3 (3), and CO-4 (4), respectively. The molar ratio of aptamer to CO was 1:5. FIG. 5(C) depicts SPR sensorgrams. The CO solution was flowed over the biochip during the dissociation. FIG. 5(D) shows the comparison of dissociation rate constants (k_{off}).

FIG. 6 depicts that an extra nonessential-nucleotide tail can significantly accelerate the aptamer-protein dissociation. FIG. 6(A) shows the structure the aptamer and hybridization region. FIG. 6(B) depicts a gel electrophoresis image. The molar ratio of apt to CO was 1:5. FIG. 6(C) displays SPR analysis of CO-mediated aptamer-protein dissociation. The concentration of CO in the washing buffer varied from 0 to 500 nM. FIG. 6(D) displays comparison of apparent dissociation rate constants (K_{off}).

FIG. 7 depicts protein release from microbead surface.

FIG. 8 depicts a schematic representation of composite synthesis and experimental results of streptavidin coated particle with biotinylated aptamer in agarose solution. FIG. 8(A) depicts a schematic representation of composite synthesis. FIG. 8(B) shows confocal transmission microscopy images of streptavidin-coated polystyrene particles (B1), aptamer-coated particles (i.e., affinity particles) (B2), and protein-coated particles (B3). FIG. 8(C) depicts confocal transmission microscopy images of the native agarose gel (C1) and the composite (C2). FIG. 8(D) depicts measurement of storage (unfilled markers) and loss (filled markers) modulus: (⋄, ♦) the native agarose gel and (□, ▪) the composite. Scale bars: 10 μm.

FIG. 9(A) depicts secondary structure of anti-PDGF-BB aptamer (filled sequence), complementary oligonucleotide (un-filled sequence), and their hybridization (The pink, blue, and green colors indicate the 10-A linker, the 36-nt essential nucleotides, and the 15-nt molecular anchorage, respectively); and FIG. 9(B) depicts gel electrophoresis analysis of intermolecular hybridization. Apt: aptamer; S-CO: scrambled complementary oligonucleotide; CO: complementary oligonucleotide.

FIG. 10(A) depicts flow cytometry analysis of affinity particles treated with the FAM-labeled COs; and FIG. 10(B) depicts confocal microscopy images of affinity particles treated with the FAM-labeled COs. Scale bars: 10 μm.

FIG. 11 depicts SPR analysis of intermolecular hybridization-mediated aptamer-protein dissociation. The dashed line is added between the association and dissociation profiles to enhance legibility. S-Apt: scrambled aptamer.

FIG. 12 depicts PDGF-BB release from different hydrogel composites in the absence of the COs.

FIG. 13 depicts microscopy images of the composite treated with the FAM-labeled COs. FIG. 16(A) depicts images by an inverted microscope and FIG. 13(B) depicts images by a confocal microscope. Red scale bars: 50 μm; white scale bars: 10 μm.

FIG. 14(A) depicts a schematic representation of protein release from the particle surface embedded in the composite in the presence of COs; and FIG. 14(B) depicts profiles of accelerated/pulsatile PDGF-BB release. The arrows show the time points of stimulating the composite with the COs.

FIG. 15 depicts secondary structures of anti-PDGF-BB aptamers with different tail compositions. The 10-nt tail is marked in blue.

FIG. 16 depicts the effect of tail variation on the aptamer-protein interaction. FIG. 16(A) depicts SPR sensorgrams and FIG. 16(B) depicts normalized equilibrium responses where the normalization was performed by dividing the equilibrium response of each aptamer by that of S1.

FIG. 17 depicts the secondary structures of anti-PDGF-BB aptamers with different mutations. The mutated nucleotides are marked in green. The 10-nt tail is marked in blue.

FIG. 18 depicts the effect of stem mutation on the aptamer-protein interaction. FIG. 18(A) depicts SPR sensorgrams FIG. 18(B) shows normalized binding responses.

FIG. 19 depicts the determination of dissociation constants (K_{D}). FIG. 19(A) shows concentration-dependent binding sensorgrams. FIG. 19(B) show equilibrium binding plot. FIG. 19(C) depicts a table showing dissociation constants.

FIG. 20 depicts particle characterization. FIG. 20(A) depicts microscopic observation of the particles in PBS. A1: streptavidin-coated polystyrene particles; A2: aptamer-coated particles; A3: aptamer-coated particles treated with PDGF-BB. Scale bar: 10 μm. FIG. 20(B) shows flow cytometry histograms.

FIG. 21 depicts hydrogel characterization. FIG. 21(A) shows particle distribution in the poloxamer hydrogel. A1: with particles; A2: without particles. Scale bar: 10 μm. FIG. 21(B) shows characterization of storage (G′) and loss (G″) modulus versus temperature. FIG. 21(C) shows characterization of storage (G′) modulus at 37° C.

FIG. 22(A) depicts PDGF-BB release from aptamer-functionalized poloxamer hydrogels. FIG. 22(B) depicts the first-day release rate as a function of K_{D} value. FIG. 22(C) demonstrates the cumulative release for 14 days as a function of K_{D} value.

FIG. 23 depicts the scanning electron microscopy (SEM) image of nano scale hydrogels functionalized with nucleic acid aptamers. Scale bars: 100 nm

FIG. 24 depicts cumulative release of PDGF-BB from nano scale hydrogels.

FIG. 25(A) depicts discovery of truncated aptamers and a schematic representation of interactions between hybridized aptamer and target: (top) no interference; (bottom) with interference. FIG. 25(B) depicts flow cytometry analysis of labeled cells; 3CON, wherein 3 means hybridization at the 3′ regions, CO means complimentary oligonucleotide, and N means the hybridization length. FIG. 25(C) depicts confocal microscope imaging of labeled cells; scale bar: 10 μm. The data demonstrate that 15-nt can be truncated at the 3′ end.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

### DETAILED DESCRIPTION OF THE INVENTION

The present invention provides a novel biocompatible affinity porous matrix composition, formulations and methods for controlling release of peptides or proteins suitable for a wide range of medical, pharmaceutical and agricultural applications. The present invention relates to a technology that provides easy-to-manufacture and reproducible compositions and formulations for peptide or protein release.

To control release of peptides or proteins from a biocompatible porous matrix (e.g., gels, polymer network, porous glass), nucleic acid aptamers are anchored to the porous matrix and the peptides or proteins whose release is to be controlled are bound to the nucleic acid aptamers with a desire affinity (e.g., high affinity). The porous matrix as a support for the nucleic acid aptamers can be a gel such as hydrogel, lipid-based gel, xenogel and organogel. The porous matrix can be a non-gel such as porous glass. The materials for the porous matrix support can be synthetic or natural materials, preferably that are biocompatible and/or biodegradable. One of ordinary skill in the art can readily select suitable materials for the porous matrix depending on the use and site for which the protein release is desired.

A composition, according to one embodiment of the present invention, comprises a porous matrix having a plurality of affinity sites provided by nucleic acid aptamers that are either directly attached to the porous matrix or indirectly anchored in the porous matrix and one or more peptides or proteins bound to the aptamers. In certain aspects of the invention, the porous matrix is a hydrogel.

Hydrogels are three-dimensional networks made of hydrophilic polymers or polymers containing hydrophilic co-polymers. Hydrogel networks are formed by the crosslinking of polymer chains via covalent bonds, hydrogen bonds, or ionic interactions, or via physical entanglement. Hydrogels can be prepared with biocompatible synthetic materials to achieve specific properties at the micro- or nano-scale level. The manipulation of the molecular weight or molecular weight distribution can be used to modulate the mechanical strength of hydrogels to satisfy different requirements. Hydrogels can be designed to modulate the porosity of the network, which can be advantageously used to control the release rate in conjunction with affinity of nucleic acid aptamers. Hydrogels can be designed in a wide variety of shapes as desired. Depending on the requirements, hydrogels can be prepared in different format of geometry such as particles, films, coatings, cylinders and slabs for in vitro and/or in vivo uses.

Hydrogels can be formed from a wide variety of biocompatible polymeric materials, including, but not limited to, polyurethane, silicone, copolymers of silicone and polyurethane, polyolefins such as polyisobutylene and polyisoprene, nitrile, neoprene, collagen, alginate and the like. For example, suitable hydrogels can be formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(-hydroxy ethyl methacrylate) and copolymers of acrylates with N-vinyl pyrrolidone, N-vinyl lactams, a poly(lactide-co-glycolide), acrylamide, polyurethanes, polyacrylonitrile, poloxamer, N-Isopropylacrylamide copolymers, poly(N-isopropylacrylamide), poly(vinyl methyl ether), poly(NIPAAm-co-PEG) and the like.

Suitable hydrogels can be formed from ABA triblock containing hydrophobic polyester (A-block) and hydrophilic polyether; triblock copolymer of poly(D,L-lactide-block-ethylene oxide-block-D,L-lactide) PLA-PEO-PLA, triblock copolymer of poly(L-lactide-block-ethylene oxide-block-L-lactide) PLLA-PEO-PLLA, triblock copolymer of poly[(D,L-lactide-coglycolide)-block-ethylene oxide-block-(D,L-lactide-co-glycolide)] PLGA-PEO-PLGA, triblock copolymer of poly[(L-lactide-coglycolide)-block-ethylene oxide-block-(L-lactide-co-glycolide)] PLLGA-PEO-PLLGA, triblock copolymer of poly[(D,L-lactide-coglycolide)-block-ethylene oxide-block-(D,L-lactide-co-glycolide)] PLGA-PEO-PLGA, triblock copolymer of poly(ε-caprolactone-block-ethylene oxide-block-ε-caprolactone) PCL-PEO-PCL, triblock copolymer of poly[(D,L-lactide-co-ε-caprolactone)-block-ethylene oxide-block-(D,L-lactide-co-ε-caprolactone)] PLC-PEO-PLC.

Hydrogels can be prepared with natural biomolecules. For example, suitable natural hydrogels can be formed from gelatin, agarose, amylase, amylopectin, cellulose derivatives such as methylcellulose, hyaluronan, chitosan, carrangenans, collagen, Gellan®, alginate and other naturally derived polymers. For example, collagen can be used to form hydrogel. Collagen can be used to create an artificial extracellular matrix that can be used as cell infiltration scaffolds for inducing tissue regeneration and remodeling. Suitable natural hydrogels also include alginate. Alginate is natural polysaccharide extracted from algae or produced by bacteria. Alginate can be a linear anionic polymer composed of 1,4-linked β-D-mannuronic acid and α-L-guluronic acid residues. In one embodiment, biocompatible alginate form hydrogels in the presence of divalent cations (e.g., Ca²⁺). Accordingly, the synthesis of alginate hydrogels can be carried out in a physiological condition where the proteins whose release is to be controlled retain their natural function. Alginate hydrogels can be used for encapsulation of functionalized aptamer-coated beads and to be used in controlled release of the protein for tissue regeneration, and protein delivery in vitro and in vivo. In another embodiment, agarose can be used to form a hydrogel.

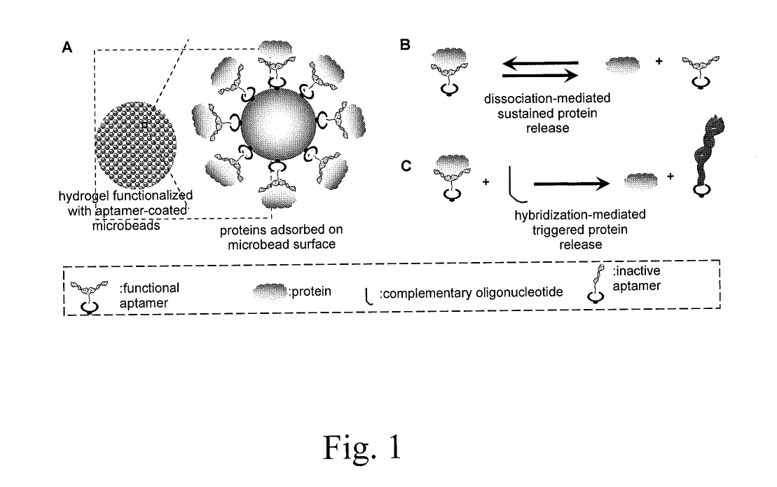

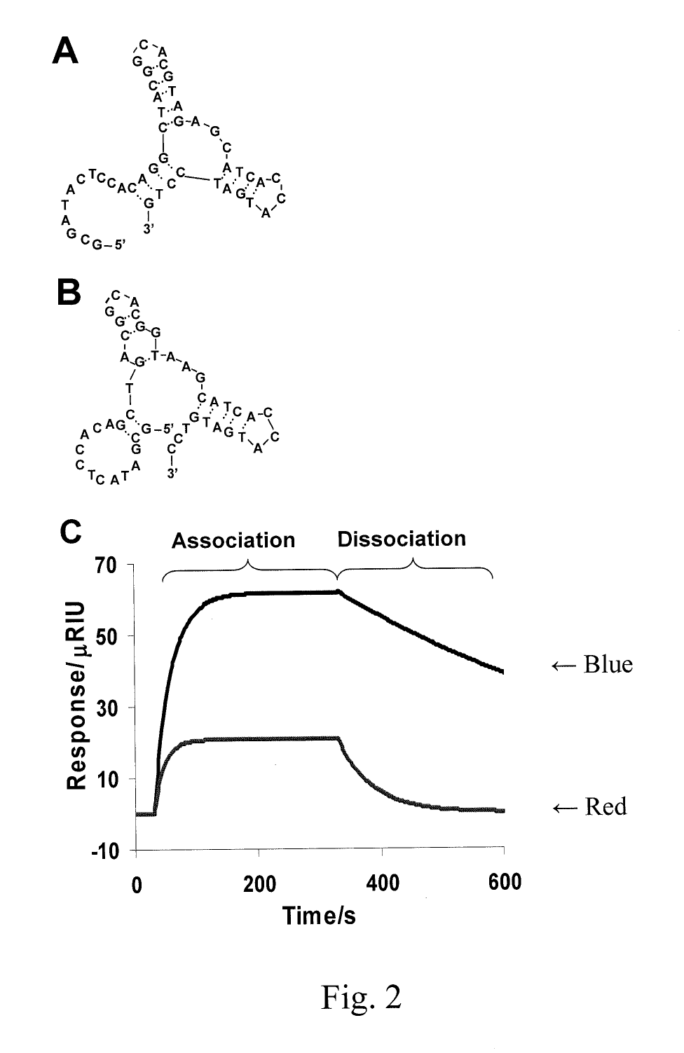

Nucleic acid aptamers are used as novel affinity sites in the porous matrices (e.g., hydrogels) for entrapping one or more peptides or proteins because of their high binding affinity and specificity to the peptide or protein of interest whose release is to be controlled. Nucleic acid aptamers could efficiently prevent rapid peptide or protein release from the porous polymeric matrix that is otherwise highly permeable. The release rate can be controlled by tuning the binding affinity of aptamers (FIGS. 1 and 2). If necessary, the nucleotide sequence or the length of the aptamers can be modified to control the affinity and the release rate of the peptides or proteins. Nucleic acid aptamers can be selected from oligonucleotide libraries for the peptide or protein of interest whose release is to be controlled. The nucleic acid aptamers can be single-stranded DNA, double-stranded DNA, RNA, or modified RNA. In one embodiment, the nucleic acid aptamers can be single-stranded nucleic acid nanostructures that are screened from DNA/RNA libraries. The technology for functionalized aptamer screening method, such as Systematic Evolution of Ligands by Exponential Enrichment (SELEX), is well known in the art and can be used to select one or more nucleic acid aptamers to be used in the invention. The nucleic acid aptamers can also have high specificity and discriminate targets on the basis of subtle structural differences. In one embodiment, aptamers have tunable stability in biological environments and their biodegradability can be controlled by the degree of nucleotide modification. Nucleic acid aptamers can have increased tolerance to harsh thermal, physical, and chemical conditions and exert little or no immunogenicity or toxicity. Nucleic acid aptamers can be synthesized with a standard chemical procedure known in the art. During the synthesis, the nucleic acid aptamers can be modified to add one or more functional groups such as acrydite, biotin, thiol, amino and the like at their 5′ and/or 3′ ends. In one embodiment of the present invention, the nucleic acid aptamers can be biotinylated to facilitate the binding of streptavidin-coated affinity particles.

The porous matrix can be functionalized with nucleic acid aptamers by either directly binding the nucleic acid aptamers to the porous polymer matrix or indirectly anchoring them within the porous polymer matrix (FIGS. 1A; 3A; 8A; and 23).

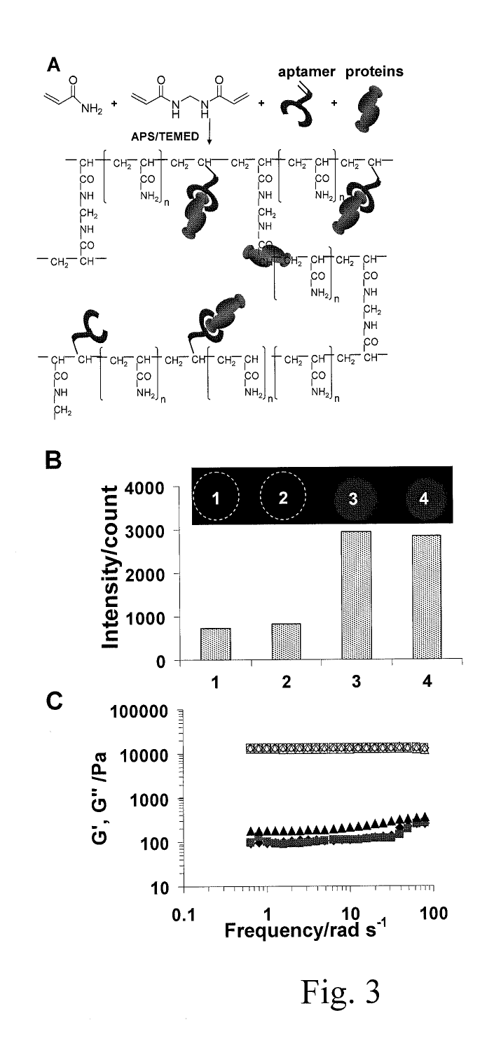

In one embodiment, the porous matrix can be functionalized with nucleic acid aptamers by directly binding the nucleic acid aptamers to the porous polymer matrix. For example, the nucleic acid aptamers bound to the peptides or proteins are reacted with the pre-gelation solution or polymeric materials to form a gel through free radical polymerization. Peptides or proteins can be incubated with the nucleic acid aptamers conjugated with an acrydite functional group at its 5′ end to achieve binding. The mixture can then be added into, for example, an acrylamide/bis-acrylamide solution. When ammonium persulfate (APS) and N,N,N′,N′-tetramethylenediamine (TEMED) are added to the mixture to initiate the free radical polymerization, the unsaturated double bond of the acrydite can enable the direct incorporation of the nucleic acid aptamer molecules into hydrogel network (FIG. 3A).

A gel-forming polymer can be also directly functionalized with the nucleic acid aptamers before the formation of a gel. For example, a gel-forming polymer, such as alginate, can be activated with N-hydroxysuccinimide (NHS). The activated alginate can then react with the nucleic acid aptamers bearing primary amino groups to form an aptamer-alginate conjugate. The conjugate is then reacted with ions (e.g., calcium ion) to form an alginate hydrogel. Another example is to apply click chemistry to functionalize hydrogel with aptamers. The click chemistry is based on the reaction between azide and alkyne. For instance, hyaluronan can be functionalized with 11-azido-3,6,9-trioxaundecan-1-amine to get azide groups attached to its side chains. The aptamer bearing an alkyne group that can be at either 5′ or 3′ end can react with the hyaluronan to get aptamer-functionalized hyaluronan

In yet another embodiment, the porous matrix gel can be prepared by providing the gel first without the nucleic acid aptamers. The gel can then be functionalized with nucleic acid aptamers bound to the proteins. For example, collagen can be crosslinked with glutaraldehyde to form a gel or sponge. Because collagen has many primary amino groups, the collagen gel or sponge can be functionalized with N-(β-maleimidopropyloxy)succinimide ester (BMPS) to obtain maleimide groups. The collagen with maleimide groups can react with nucleic acid aptamers bearing thiol groups to synthesize an affinity gel or sponge functionalized with the aptamer.

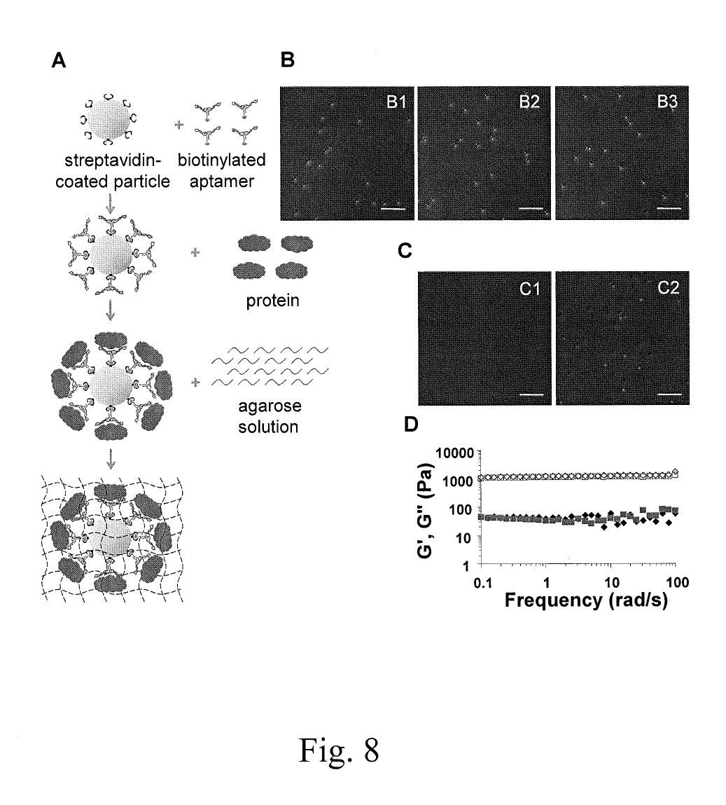

The porous matrix can be functionalized with nucleic acid aptamers by indirectly anchoring the nucleic acid aptamers within the porous polymer matrix. The porous matrix gel can be prepared by conjugating the aptamers with particles such as micro- or nano-beads. The peptides or proteins can be loaded onto the nucleic acid aptamers. The functionalized affinity particles coated with the aptamers bound to the peptides or proteins are mixed with the gel-forming polymer to allow polymerization. By attaching to the affinity particles embedded in the porous gel, the aptamers are indirectly anchored in the matrix of the gel (FIG. 8A).

Suitable particles for use with this invention can be small regularly or irregularly shaped biocompatible particles, which can be solid, porous or hollow to which aptamers can be affixed by coating the particles with the aptamers or affixing them covalently or by other affixation techniques. Preferably, the particles are microbeads or nanobeads. Generally, microbeads will have an average diameter of between from about 1 and to about 1000 μm and nonobeads will have an average diameter of less than 1 μm. In some embodiments, the microbeads or nanobeds will have a generally uniform diameter. Additionally, in particularly preferred embodiments, the particles can be biodegradable, for example, particles made from poly (lactide-co-glycolide) or a hydrogel particle. The affinity particles can be used to coat, tether, attach, or conjugate one or more nucleic acid aptamers having high specificity to peptides or proteins whose release is to be controlled from the porous matrix and to anchor them in the porous matrices (e.g. hydrogel). The affinity particles are suitable for attaching, coating, conjugating, tethering or coupling with a ligand, including, but not limited to, protein, peptide, antibody, streptavidin, protein, antigen, nucleic acid (e.g., DNA, RNA, nucleic acid aptamers) or other biomolecules. In some embodiments, the particles can be conjugated, coated, tethered, bonded, or coupled to the nucleic acid aptamers. In certain embodiments, the particles are coated with streptavidin. When the particles are hydrogels, the particles can be also used as the porous matrix described above for the aptamers that are directly attached to the particles.

Peptides or proteins bound to the nucleic acid aptamers can vary depending on the purpose for which the protein release is desired. In some embodiments, peptides or proteins can have a therapeutic value in treating a disease or condition in a subject. Such diseases or disorders include, but are not limited to, pancreatic cancer, papillary thyroid carcinoma, ovarian carcinoma, human adenoid cystic carcinoma, non small cell lung cancer, secretory breast carcinoma, congenital fibrosarcoma, congenital mesoblastic nephroma, acute myelogenous leukemia, psoriasis, metastasis, cancer-related pain and neuroblastoma, autoimmune diseases, inflammatory diseases, bone diseases, metabolic diseases, neurological and neurodegenerative diseases, cancer, cardiovascular diseases, respiratory diseases, allergies, asthma, hormone related diseases, benign and malignant proliferative disorders, diseases resulting from inappropriate activation of the immune system and diseases resulting from inappropriate activation of the nervous systems, allograft rejection, graft vs. host disease, diabetic retinopathy, choroidal neovascularization due to age-related macular degeneration, psoriasis, arthritis, osteoarthritis, rheumatoid arthritis, synovial pannus invasion in arthritis, multiple sclerosis, myasthenia gravis, diabetes mellitus, diabetic angiopathy, retinopathy of prematurity, infantile hemangiomas, bladder and head and neck cancers, prostate cancer, breast cancer, ovarian cancer, gastric and pancreatic cancer, psoriasis, fibrosis, atherosclerosis, restenosis, autoimmune disease, allergy, respiratory diseases, asthma, transplantation rejection, inflammation, thrombosis, retinal vessel proliferation, inflammatory bowel disease, Crohn's disease, ulcerative colitis, bone diseases, transplant or bone marrow transplant rejection, lupus, chronic pancreatitis, cachexia, septic shock, fibroproliferative and differentiative skin diseases or disorders, central nervous system diseases, neurodegenerative diseases, Alzheimer's disease, Parkinson's disease, disorders or conditions related to nerve damage and axon degeneration subsequent to a brain or spinal cord injury, acute or chronic cancer, ocular diseases, viral infections, heart disease, lung or pulmonary diseases or kidney or renal diseases and bronchitis. One of ordinary skill in the art would appreciate that the present invention can be applied to any situation in which peptides or proteins must be delivered and released in a reproducible and controlled manner to treat a disease or condition or to create an artificial scaffold for inducing tissue regeneration and remodeling to recapitulate the features of extracellular matrices in a mammal. In some embodiments, peptides or proteins whose release is to be controlled have one or more useful functions in biological research.

Complementary oligonucleotides (COs) that bind or hybridize to nucleic acid aptamers can be used as molecular triggers that regulate, modulate or accelerate the release of proteins from nucleic acid aptamers in the hydrogel network. For example, the release kinetics can be modulated by introducing the complementary oligonucleotides that interfere with the interactions between the aptamers and the proteins into the porous matrix (FIG. 1C). The nucleic acid aptamers in some case are modified so that they contain extra non-essential nucleotides that facilitates the binding of complementary oligonucleotides. The complementary oligonucleotides can be about 5- to about 30-nucleobase long. The complementary oligonucleotides can bind to an aptamer sequence that overlaps between the essential and non-essential sequence. When the aptamer-functionalized polymeric porous matrix loaded with peptides or proteins is triggered with the complementary oligonucleotides, a significant amount of proteins can be released in a short period of time. The amount can be delicately tuned by the triggering time and triggering dose. In certain embodiments, the nucleic acid aptamers and/or complementary oligonucleotides can be fluorescently labeled for detection.

According to certain embodiments of the invention, the peptides or proteins can be loaded onto the aptamer-functionalized particles before or after the polymerization and gelation of the porous matrix, for example, a hydrogel. Preferably, the aptamers can be first bound to the peptides or proteins to be released (“protein-functionalized aptamers”) and the protein-functionalized aptamers can be transferred into a pre-gelation polymer solution that forms a porous matrix or the affinity particles that are to be embedded in the porous matrix. In yet another embodiment, the proteins can be loaded on the affinity particles during or after the gelation by introducing the proteins to a polymeric solution or the porous matrix of hydrogel functionalized with nucleic acid aptamers.

According to one embodiment of the present invention, a method of controlling peptide or protein release from a porous matrix (e.g., hydrogel) is also provided. For example, a hydrogel comprising a plurality of affinity sites provided by nucleic acid aptamers that bind to peptides or proteins can be used in the method. The peptides or proteins are bound to the high affinity nucleic acid aptamers at a predetermined affinity suitable for a desired release rate. In a sustained protein release mode, the protein release kinetics of the hydrogel functionalized with nucleic acid aptamers is slower as compared with that of the hydrogels that are not functionalized with the aptamers. The release kinetics of the peptide or protein of interest can be tuned by modulating the binding affinity of nucleic acid aptamers selected for a formulation of that protein. The affinity can be also modulated by adding new non-essential nucleotides at 5′ and/or 3′ end of the aptamer or by mutating the existing aptamer sequence. The peptide or protein release from the porous matrix can be further modulated by the use of complementary oligonucleotides that accelerate aptamer-protein dissociation. In some embodiment, the proteins are released for a defined time period and/or at a predetermined amount.

Methods for making a formulation for controlled protein release are also provided in the present invention. To make a formulation that comprises a porous matrix that contains affinity sites of aptamers indirectly anchored to the matrix, the method involve the steps of (a) coating or conjugating nucleic acid aptamers on the surface of micro- or nano-particles, (b) contacting said nucleic acid aptamer-coated particles with proteins having binding affinity to said nucleic acid aptamers and whose release is to be controlled, (c) allowing said proteins to bind said one or more nucleic acid aptamers, (d) transferring the aptamer-coated beads saturated with said proteins into a pre-gelation polymer solution, and (e) forming a gel. In an alternative method, the nucleic acid aptamers are directed bound to the porous matrix by providing the nucleic acid aptamers having a functional group for direct conjugation with the porous matrix. In such a method, the affinity particles are not involved in anchoring the aptamers into the matrix. In some embodiments, the gel is formed under a physiological condition in which the peptides or proteins retain original function. In still another embodiment, the gel is a biocompatible hydrogel. In certain embodiments, the hydrogel are naturally occurring polymers, including, but not limited to, agarose, collagen, alginate, methylcellulose, hyaluronan and the like.

According to one embodiment of the present invention, a method for delivering peptides or proteins to a subject in need of such peptides or proteins is also provided. The method comprises providing a formulation comprising a porous matrix (e.g., hydrogel) having affinity sites provided by nucleic acid aptamers having high affinity to the peptides or proteins to be delivered. The subject can be a human patient in need of the peptides or proteins for treatment in, for example, pediatric short stature, Turner's syndrome, inflammatory diseases such as rheumatoid arthritis, or chronic renal failure. For short stature, the protein can be human growth hormone (hGH). If the subject is a dairy cow, the protein can be bovine somatotropin. The release systems of the present invention can be applied to virtually any area that needs sustained or controlled peptide or protein release. The peptides or proteins can include, but are not limited to, growth hormones, anti-inflammatory biologics such as anti-TNF-α, anti-IL-1, anti-IL-6 antibodies or fusion proteins useful in treating, for example the diseases or conditions described above. The peptides for proteins involved in metabolic processes can be used to treat a metabolic disease or condition in a subject.

One of the challenges in tissue engineering and regenerative medicine is the creation of novel materials to mimic key features of extracellular matrices. Natural extracellular matrices play multiple, complex, and dynamic roles in tissues. They provide cells with an intricate microenvironment that is comprised of insoluble macromolecules (e.g., proteoglycans), soluble signal molecules (e.g., growth factors), and adhesion ligands (e.g., fibronectin). Based on the communication with these effectors at the nanoscale level, cells acquire essential biophysical and biochemical cues as well as mechanical supports. Thus, multiple signal pathways can be triggered to turn on or off in the cells. As a result, the coordination of numerous signals from the extracellular microenvironment can determine whether a cell will undergo proliferation, migration, differentiation, apoptosis, or other functions. Therefore, biomaterials recapitulating the features of natural extracellular matrices would have capabilities of instructing cell behaviors for tissue engineering, repair and regeneration. Accordingly, the invention also provides a novel approach for the synthesis of biomimetic materials to address the aforementioned challenges in the field of tissue engineering and regenerative medicine by providing a porous matrix capable releasing peptides or proteins in a controlled manner

A kit for delivering proteins in a controlled manner is also provided in the present invention. The kit comprises a container containing a formulation comprising a hydrogel having affinity sites comprising nucleic acid aptamers as described herein. For example, the aptamers are bound directly to the matrix or anchored indirectly via affinity particles embedded in the matrix. In another embodiment, the container can contain a pre-gelation solution for the hydrogel and the apatamers can be supplied in a separate container. In such a case, the user performs the mixing and polymerization procedures. The peptides or proteins whose release is to be controlled can be supplied in the kit or can be supplied separately. The proteins to be released are bound to the nucleic acid aptamers before, during, or after the gelation or polymerization. In one embodiment, the kit further comprises complementary oligonucleotides that hybridize with the one or more high affinity nucleic acid aptamers. The hybridization of the complementary oligonucleotides with the high affinity nucleic acid aptamer causes acceleration of protein release from the hydrogel. Instructions for making and using the present invention described herein are also provided in the kit.

Preparation of controlled-release formulations comprising proteins is simple and reproducible. Proteins can be simply mixed with aptamer-tethered micro-/nano-beads, pre-gelation polymer, or porous matrix. Further, the protein release from a formulation can be decoupled from gel degradation. Thus, the release does not rely on degradation of hydrogels providing an advantage over the conventional approaches in that the mechanical properties and integrity of hydrogels is maintained during the desired time period of administration and controlled agent release. Thus, the composition and method of the present invention provide more flexibility and safety of the controlled release dosage form. The mechanical properties of dosage forms are among the important factors influencing the efficacy and safety of controlled drug release systems.

One of ordinary skill in the art will be able to determine a desired peptide or protein release rate or pulsatile release pattern empirically or by a mathematical model. Methods of modeling or determining a peptide or protein release rate are described in Soontornworajit et al. “Hydrogel functionalization with DNA aptamer for sustained PDGF-BB release” Chem. Commun., (2010) 46:1857-1859; and Soontornworajit et al., “Hydrogel functionalization with aptamers for sustained protein release” Chem. Commun., Supporting Information (2010) 46:S1-S7, the teachings of which are incorporated herein by reference.

“Aptamer Database” “GenBank” and “SELEX_DB” are comprehensive and continuously updated databases and contain extensive information on potential aptamers that can be used in the present invention. These resources are readily available to one of ordinary skill in the art and useful in selecting appropriate aptamers to be implemented in the present invention.

As used herein, the term “aptamer” refers to nucleic acid that binds to a peptide or protein with high specificity and affinity and is generated by in vitro selection. The term “nucleic acid aptamers” includes, but is not limited to, RNA, modified RNA, single-stranded DNA or double-stranded DNA.

The term “growth factors” includes any cellular product that modulates the growth or differentiation of other cells, particularly connective tissue progenitor cells. The growth factors that can be used in accordance with the present invention include, but are not limited to, human growth factor (hGF), members of the platelet-derived growth factor (PDGF) family, including PDGF-AB, PDGF-BB and PDGF-AA; endothelial growth factors (EGFs) such as vascular endothelial growth factor (VEGF); members of the fibroblast growth factor family, including acidic and basic fibroblast growth factor (FGF-1 and FGF-2) and FGF-4; members of the insulin-like growth factor (IGF) family, including IGF-I and -II; the TGF-β superfamily, including TGF-β1, 2 and 3; angiogenin(s); endothelins; hepatocyte growth factor and keratinocyte growth factor; members of the bone morphogenetic proteins (BMP's) BMP-1, BMP-2, BMP-3, BMP-5 and BMP-7, BMP-14; HBGF-1 and HBGF-2; growth differentiation factors (e.g., GDF-5), members of the hedgehog family of proteins, including indian, sonic and desert hedgehog; ADMP-1; members of the interleukin (IL) family, including IL-1 thru IL-6; and members of the colony-stimulating factor (CSF) family, including CSF-1, G-CSF, and GM-CSF; and isoforms thereof.

As used herein, the term “subject” or “patient” encompasses mammals and non-mammals that are need of one or more peptide or protein(s) to be released by the present invention. Examples of mammals include, but are not limited to, humans, apes, monkeys, cattle, horses, sheep, goats, swine; rabbits, dogs, cats, rats, mice, guinea pigs, and the like. Examples of non-mammals include, but are not limited to, reptiles, birds, fish and the like.

As used herein, the term “administration” or “administering” of the formulation refers to providing the formulation of the invention to a subject in need of treatment.

As used herein, the term “acceptable” with respect to a formulation, composition or ingredient, as used herein, means having no persistent detrimental effect on the general health of the subject being treated.

As used herein, the terms “effective amount” or “therapeutically effective amount” refer to a sufficient amount of a formulation described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a formulation as disclosed herein required to provide a clinically significant decrease in disease symptoms. The term also includes within its scope amounts effective to enhance normal physiological function. As used herein, “therapeutically effective amount” will vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

It is to be understood that the term “proteins” encompasses “peptides,” if “peptides” are not specifically recited along with “proteins.”

### EXAMPLES

The invention is further described, for the purpose of illustration only, in the following examples.

### Example 1

### Hydrogel Functionalization with DNA Aptamers for Sustained PDGF-BB Release

### Materials and Methods

### Reagents.

N-ethyl-N-(3-diethylaminopropyl)carbodiimide (EDC), N-hydroxysuccinimide (NHS), Tween 20, ammonium persulfate (APS), N,N,N′,N′-tetramethylenediamine (TEMED), and a premixed solution of acrylamide and bis-acrylamide (40%; 29:1) were purchased from Fisher Scientific (Suwanee, Ga.). The anti-PDGF-BB aptamer (5′-/Acrydite/GC GAT ACT CCA CAG GCT ACG GCA CGT AGA GCA TCA CCA TGA TCC TG-3′; SEQ ID NO:1) and its control aptamer (5′-/Acrydite/GC GAT ACT CCA CAG CTG ACG GCA CGG TAA GCA TCA CCA TGA TGT CC-3′; SEQ ID NO:2) were purchased from Integrated DNA Technologies (Coralville, Iowa). The 10-nt tail sequence is marked in blue. Recombinant Human PDGF-BB was purchased from R&D Systems (Minneapolis, Minn.). Bovine serum albumin (BSA) was purchased from Invitrogen (Carlsbad, Calif.). Human PDGF-BB ELISA development kit was purchased form PeproTech (Rocky Hill, N.J.). Diammonium 2,2′-azinobis(3-ethylbenzothiazoline-6-sulfonate) (ABTS) was purchased from Sigma-Aldrich.

### Secondary Structure Prediction.

The secondary structures of the aptamers were generated with the program RNAstructure version 4.6 (http://rna.urmc.rochester.edu/rnastructure.html). Of the secondary structures generated, the most stable ones with the lowest free energies were presented.

### Surface Plasmon Resonance Analysis

The molecular interaction between PDGF-BB and the aptamers were studied by SPR spectrometry (SR7000DC, Reichert Analytical Instrument, Depew, N.Y.). PDGF-BB was immobilized onto a sensor chip via amide synthesis. An aqueous solution of NHS (0.0115 g/ml) and EDC (0.038 g/ml) was flowed over the sensor chip at a flow rate of 30 μL/min. After the EDC/NHS-mediated activation of the biochip surface for 10 min, 10 μg/mL of PDGF-BB solution (pH 8.5) was flowed over the chip surface for protein immobilization. Prior to the molecular recognition analysis, the sensor chip was equilibrated for 30 min with a phosphate-buffered saline (PBS) solution containing 0 05% Tween 20. During the binding analysis, 500 nM of binding solution containing either anti-PDGF-BB aptamer or its control was flowed over the biochip at 30 μL/min. The biochip was regenerated with 1 M of NaCl. The binding analysis was repeated twice to confirm the reproducibility. The equilibrium dissociation constant (KD) was calculated with direct curve fitting of the sensorgrams using the software provided by the manufacturer.

### Hydrogel Synthesis.

To prepare aptamer-functionalized hydrogels, PDGF-BB was incubated with the aptamer at a molar ratio of 1:625 in 50 μL of PBS (pH 7.4) for 30 min. The mixture was then added into a 10% of acrylamide/bis-acrylamide solution to prepare 1000 μL of solution. After gentle mixing of the acrylamide solution, 1 μL of APS solution (0.43 M) and 1 μL of TEMED solution (20%) were added sequentially. 1000 μL of solution was immediately transferred into a 0.5 mL cylindrical mold made from an insulin injection syringe. The polymerization was carried out for 3 h at room temperature. The molar ratio of aptamer to acrylamide monomer was fixed at ˜1:2,800,000. Native hydrogels were prepared with the same protocol except the addition of the aptamer. The hydrogels were cut into small pieces (50 μL/each) for a releasing study experiment.

### Examination of Aptamer Incorporation.

Gel electrophoresis has been used to examine the efficiency of oligonucleotide incorporation into hydrogels because free oligonucleotides could be removed from the hydrogels during electrophoresis. Therefore, we performed two gel electrophoresis experiments to examine aptamer incorporation. In the first experiment, 50 μL polyacrylamide hydrogels were subjected to gel electrophoresis for 90 min with a Bio-Rad Sub-Cell GT agarose gel electrophoresis system, stained with ethidium bromide for 30 min, and finally incubated in 1.5 mL PBS buffer on a shaker for another 30 min to remove free unintercalated ethidium bromide. In the second experiment, we first synthesized a piece of native polyacrylamide gel with loading wells. After the preparation of polyacrylamide solutions with acrydited aptamers or non-arydited aptamers, we then transferred the polyacrylamide solutions into the loading wells. The final gels were subjected to vertical nucleic acid electrophoresis with a Bio-Rad Mini-PROTEAN tetra cell and stained by ethidium bromide. Gel images were captured with a Bio-Rad GelDoc XR system (Hercules, Calif.). The intensities of free-aptamer bands and aptamer-functionalized hydrogels were analyzed with the Quantity One software (Bio-Rad, Hercules, Calif.) and used as an indicator to qualitatively determine the percentage of free DNA aptamers that were unincorporated into the hydrogel network.

### Rheology Characterization.

The storage and loss moduli of hydrogels were measured with an AR-G2 rheometer (TA Instruments, New Castle, Del.). The gels were prepared as circular discs of 20 mm in diameter and 1.5 mm in thickness. The samples were placed between two plates covered by a humidity chamber. The temperature was set at 37° C. for all experiments. The gap was adjusted until the normal force reached 0.3 N. To confirm that the measurement was in the linear viscoelastic regime, a stress sweep was performed by varying the oscillation stress from 0.01 to 1000 Pa at a fixed frequency of 6 rad/s. Frequency sweep experiments were performed from 0.6 to 90 rad/s at 1 Pa oscillation stress.

### Sustained Release Study.

For each type of hydrogel, we prepared 14 hydrogel samples. The samples were incubated at 37° C. in a 1.5 mL centrifuge tube containing 450 μL of release medium. The release medium was a mixture of PBS and 0.5% BSA. The shaking rate was 70 RPM. At the predetermined time points (1, 6, 12, 24, 48, 96, and 144 hr), two hydrogel samples were taken out of the tubes to stop the release. At the end of the release experiments, native hydrogels were minced and PDGF-BB was eluted from the gels to calculate the total amount of PDGF-BB. The released PDGF-BB was quantified by human PDGF-BB ELISA.

### ELISA Analysis.

ELISA microplate was coated with 100 μL of anti-hPDGF-BB antibodies (0.5 μg/mL in PBS) at 4° C. for overnight. The wells were washed by 300 μL of washing buffer (0.05% tween 20 in PBS) for 4 times. The wells were subsequently blocked by 1% BSA in PBS for 2 hours and then were washed by 300 μL of washing buffer for 4 times. Then, 100 μL of releasing samples and standard proteins solutions, reconstituted in a diluent (0.1% BSA and 0.05% tween 20 in PBS), were incubated in the coated wells for 2 hours before being discarded. The wells were washed with 300 μL of washing buffer for 4 times. 100 μL of biotinylated antibodies (0.25 μg/mL in the diluent) were added into the wells and incubated for 2 hours. The wells were washed with 300 μL of washing buffer for 4 times. 100 μL of avidin-HRP conjugate were added into the wells and incubated for 30 min. After discarding avidin-HRP conjugate, the wells were washed with 300 μL of washing buffer for 4 times. 100 μL of substrate were added and incubated for 30 minutes. Subsequently, absorptions at 405 and 650 nm were recorded by Synergy HT Multi-Mode microplate reader (BioTeK, Winooski, Vt.). The amount of PDGF-BB release was calculated by subtracting absorbance at 650 nm from absorbance at 405 nm. All experiments were performed in duplicate.

### Results

Polyacrylamide gel and anti-platelet-derived growth factor-BB (PDGF-BB) aptamer were used as a model system. The anti-PDGF-BB aptamer was originally selected from a DNA library. The aptamer sequence used here is composed of its truncated 36-nucleotide (nt) format and a 10-nt tail attached to the 5′ end. The truncated 36-nt aptamer is used to bind to PDGF-BB, whereas the 10-nt tail is used to enhance molecular flexibility. The structures of anti-PDGF-BB aptamers with higher and lower affinity are shown in FIGS. 2A and B, respectively. The binding functionality was evaluated with surface plasmon resonance (SPR) analysis, which directly provides information on the binding affinity. The K_{D} values of the higher-affinity and lower-affinity aptamers are 25 nM and 220 nM, respectively (FIG. 2C).

To functionalize polyacrylamide gel, the aptamer was conjugated with an acrydite functional group at its 5′ end during its chemical synthesis (FIG. 3A). Thus, when ammonium persulfate (APS) and N,N,N′,N′-tetramethylenediamine (TEMED) were added into the mixture of acrydite-modified aptamer, acrylamide, and bis-acrylamide to initiate free-radical polymerization. The unsaturated double bond of the acrydite would then enable the incorporation of aptamer molecules into hydrogel networks (FIG. 3A).

To test the feasibility, polyacrylamide gels were subjected to electrophoresis, stained with ethidium bromide (EtBr), and examined by an imaging system. If the gels were functionalized with the aptamers, the aptamers would stay in the gels during the electrophoresis. Thus, EtBr stain would appear on the gels. FIG. 3B demonstrates that the acrydite-modified aptamer molecules were successfully incorporated into the hydrogels during polymerization. It was also observed that not all DNA aptamers were incorporated into the polyacrylamide hydrogel. The analysis of free aptamer band indicates that approximately 7.3% of DNA aptamers were free molecules after polymerization. Because mechanical strength is an important factor influencing the performance of a drug delivery system, the storage and loss moduli of polyacrylamide gels were characterized with and without aptamers (FIG. 3C). The results indicated that the incorporation of aptamers did not significantly affect the mechanical properties of polyacrylamide hydrogels. This is reasonable because the molar ratio of aptamer to acrylamide was very small and only about 1:2,800,000,

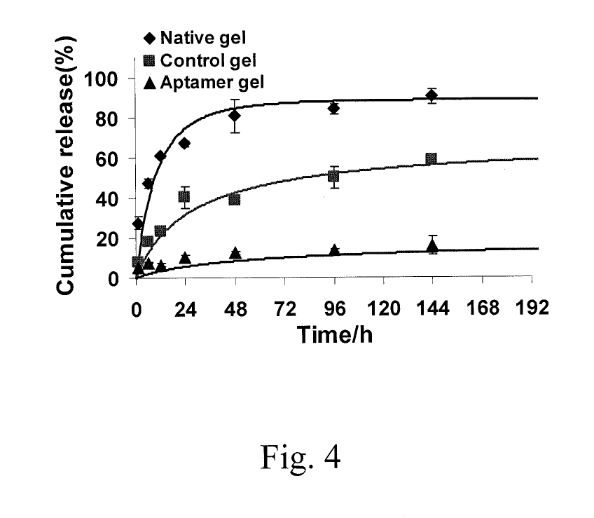

After showing the feasibility of incorporating aptamers into hydrogels, aptamer-functionalized hydrogels containing PDGF-BB were synthesized to investigate the sustained release kinetics of PDGF-BB. PDGF-BB and its aptamer were mixed at a molar ratio of 1:625 and incubated at room temperature for 30 minutes to allow for sufficient molecular recognition and complex formation. The mixture was then transferred to a solution containing acrylamide and bis-acrylamide (29:1). After gentle mixing and the addition of APS and TEMED, solutions were immediately cast into molds to allow for gel crosslinking. To remove free proteins that did not form complexes with conjugated aptamers, the gels were washed for three times with the release medium. The percentages of released PDGF-BB from native hydrogel, lower-affinity hydrogel, and higher-affinity hydrogel during washing were 4.3%, 4.2%, and 5.4%, respectively. The release of proteins from affinity hydogels during the washing step are likely attributed to the existence of unincorporated aptamers. After washing, the hydrogels were subjected to the sustained-release tests. Native hydrogels showed a significant burst-release effect (FIG. 4). During the first 24 hour, approximately 70% of PDGF-BB was released. Clearly, PDGF-BB was released from native hydrogel very rapidly. In contrast, gels functionalized with higher-affinity aptamers significantly improved the capability of sustained release (FIG. 4). The initial 24-hour release was significantly decreased from ˜70% to ˜10%. After that, ˜6% of PDGF-BB was slowly released during the next 120 hours. The release of PDGF-BB from the hydrogels with lower-affinity aptamers was slower than that from the native hydrogels, but faster than that from the hydrogels with higher-affinity aptamers (FIG. 4). The cumulative amounts released from the native hydrogel, lower-affinity hydrogel, and higher-affinity hydrogel were 428±18, 277±4, and 75±22 pg, respectively

Affinity molecules such as antibodies, antibiotics and aptamers have been used to design stimuli-sensing systems. Different from these systems, this study aims at exploring a new sustained-release system that can, in principle, be used to slowly release any molecule of interest. Importantly, the release mechanism is dependent on both specific binding and diffusion instead of bulk decomposition. Thus, unlike the stimuli-sensing systems that undergo simultaneous bulk decomposition and loss of mechanical strength during stimulus, the structural integrity of the porous matrix protein release system described herein does not need to be sacrificed during the sustained or controlled release. This new release system can be tuned to achieve on-demand release kinetics. The aptamer-functionalized hydrogels exemplified herein can be also applied to the controlled release of peptides and oligonucleotides.

In conclusion, this study successfully demonstrated a novel peptide or protein release porous matrix system by using the anti-PDGF-BB aptamer and polyacrylamide hydrogel as a model. This system holds great potential for the development of new pharmaceutical formulations and the applications of protein release for regenerative medicine.

### Example 3

### Modulating Aptamer-Protein Interactions with Complementary Oligonucleotides

One important advantage of the present invention is achieved with the application of complementary oligonucleotides as a molecular trigger to control aptamer-protein interactions and, therefore, release kinetics.

To demonstrate whether complementary oligonucleotides can efficiently compete with proteins to bind aptamers, PDGF-BB and its different aptamer formats were used as a model system.

In this example, both aptamers and complementary oligonucleotides are single-stranded nucleic acids. Their nucleotides can form self-assembled base pairs via intramolecular hybridization. When these two molecules are mixed together, intermolecular hybridization competes with intramolecular hybridization to generate double-stranded structures. Moreover, in the presence of proteins, complementary oligonucleotides compete with the binding domains of target proteins. These two competition reactions determine whether complementary oligonucleotides can efficiently modulate the aptamer-protein interactions.

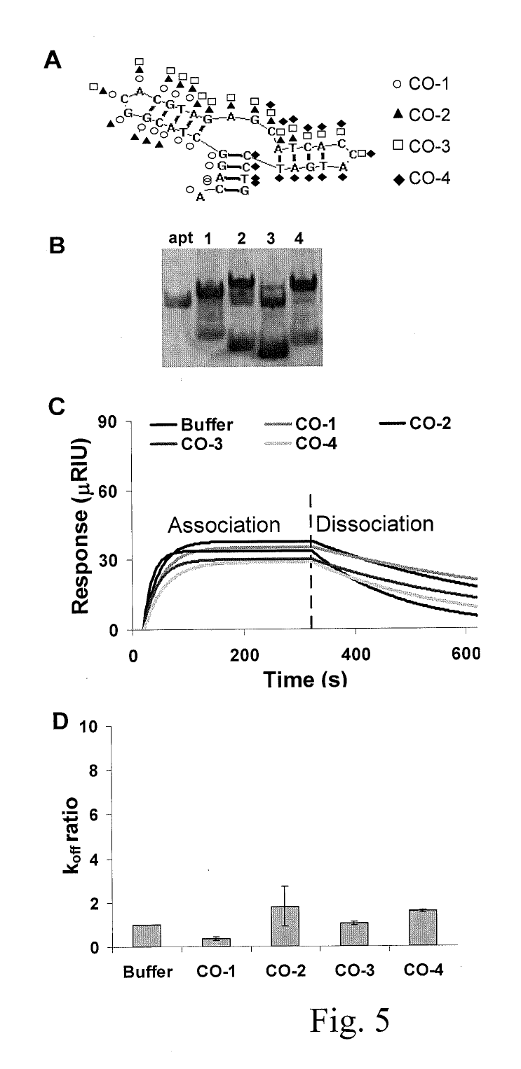

The effects of hybridization region on intermolecular hybridization and aptamer-protein dissociation were determined (FIG. 5A). The aptamer was a truncated format of anti-PDGF-BB aptamer with 36 essential nucleotides. The length of hybridized essential nucleotides was fixed at 16. FIG. 5B depicts the efficiency of hybridization between aptamers and complementary oligonucleotides. In this experiment, the aptamers and the complementary oligonucleotides were mixed together at 37° C. for 10 minutes followed by gel electrophoresis analysis. The CO-3 demonstrates a weaker capability of hybridizing with the aptamer presumably because of its stronger intramolecular hybridization. However, the overall gel image shows that the aptamer could hybridize with the complementary oligonucleotides at 37° C.

PDGF-BB was immobilized on biochip surface and aptamer-protein interactions were analyzed in the absence or presence of complementary oligonucleotides using surface plasmon resonance (SPR) spectroscopy (an efficient tool to analyze molecular interactions). As shown in FIGS. 5C & 8D, the complementary oligonucleotide-mediated dissociation of 36-nt aptamers from proteins was not significantly improved, which is different from the gel electrophoresis analysis. The difference is believed due to molecular competition between proteins and complementary oligonucleotides. For the gel electrophoresis experiment, no proteins were involved. However, for the SPR experiment, aptamers, complementary oligonucleotides, and proteins were all included in the system. Therefore, the results suggested that without any modification, it is difficult for complementary oligonucleotides to hybridize with aptamers once aptamer-protein complexes are formed.

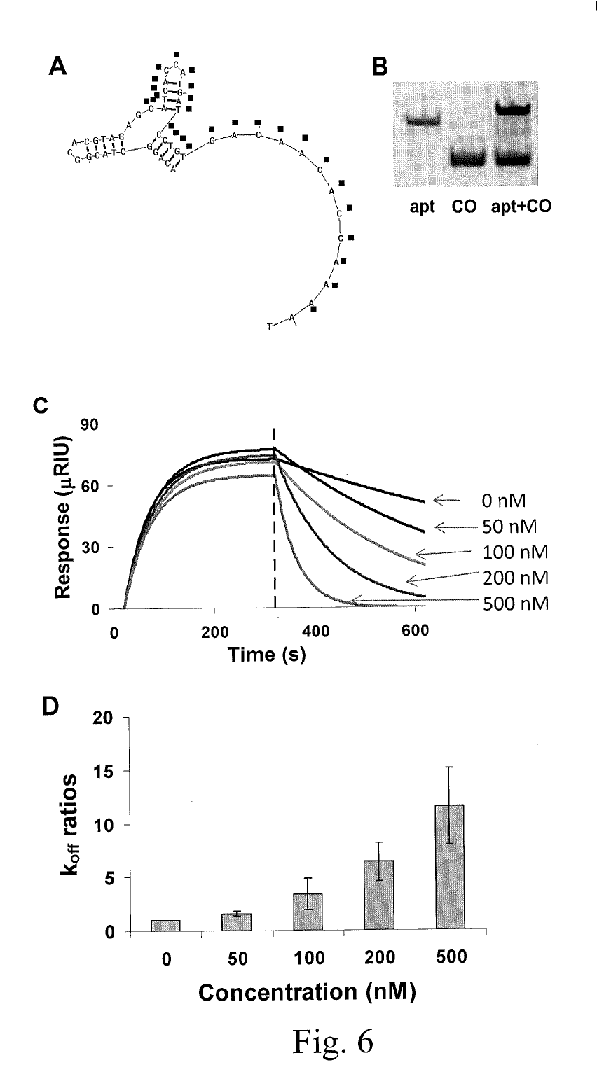

To solve this problem, the 36-nt aptamer was modified by attaching an extra 15-nt tail at the 3′ end (FIG. 6A) to enhance the competitiveness of complementary oligonucleotides in hybridizing with the protein-bound aptamers. The hypothesis was that the extra nucleotide tail could function as an anchor for the complementary oligonucleotide to associate with the aptamer. Thus, it would be easier for the complementary oligonucleotides to compete with the bound protein. The gel electrophoresis analysis showed that the aptamer with the tail hybridized with the complementary oligonucleotide (FIG. 6B). Importantly, the SPR analysis showed that the modified aptamer could bind to PDGF-BB, and that the complementary oligonucleotide significantly accelerated the dissociation of the aptamer from the protein (FIGS. 6C & D). To better understand the capability of complementary oligonucleotides in controlling the aptamer-protein interactions, the effect of CO concentration on the apparent dissociation rate constant was determined. The dissociation was significantly increased with the concentration (FIGS. 6C & D). Taken together, the results showed that intermolecular hybridization can modulate the aptamer-protein interactions.

### Example 4

### Aptamer-Coated Microbeads for Protein Adsorption

One aspect of the present invention is the entrapment and release of protein drugs in the hydrogels. The feasibility of the use of aptamer-coated microbeads to adsorb, immobilize and release proteins was assessed. When the microbeads are physically mixed with polymer solution to form hydrogels, the proteins are entrapped and incorporated into the hydrogel networks. Because the overall procedure only involves physical mixing, the method could potentially be applied to any type of hydrogel including those hydrogels that cannot be easily functionalized with chemical modification.

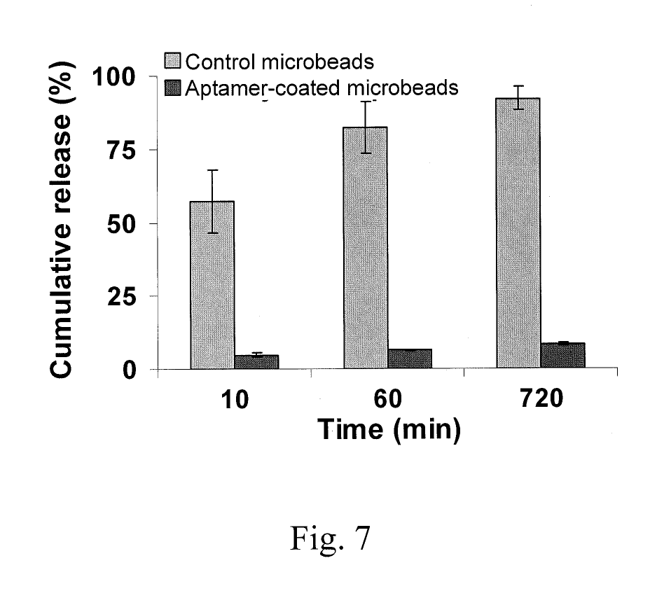

An experiment to test protein adsorption and dissociation on microbead surface was carried out. The model microbeads are streptavidin-functionalized polystyrene microbeads purchased from Spherotech (Lake Forest, Ill.). The anti-PDGF-BB aptamer was chemically modified with biotin. The biotinylated aptamers was mixed with microbeads to coat the aptamers on microbead surface. Free aptamers were removed through centrifugation. The purified aptamer-coated microbeads were incubated in protein solution for 30 minutes to allow sufficient protein binding to microbead surface. After the incubation, the microbeads were subjected to cyclic washing and centrifugation at 10, 60, and 720 minutes. Each time, half of the supernatant was collected and an equal volume of fresh binding buffer was added into the centrifuge tubes. As shown in FIG. 7, the aptamer-coated microbeads could efficiently adsorb proteins on the surface and prevent proteins from rapid dissolution into the bulk buffer.

### Example 5

### A Hybrid Particle-Hydrogel Composite for Oligonucleotide-Mediated Pulsatile Protein Release

Pulsatile release is the rapid release of a certain amount of molecules during a defined time period. It is a common phenomenon found in the human body. For instance, a number of hormones (e.g., growth hormones) are secreted from regulatory cells in a pulsatile manner to de- or re-sensitize target cells. This naturally occurring mechanism has been mimicked for the development of pulsatile protein release systems to improve therapeutic efficacy and minimize the undesired toxicity of protein drugs. One promising material system for use in pulsatile protein delivery is hydrogels.

Hydrogels can be synthesized in biocompatible conditions and have a tunable viscoelasticity and structural similarity to natural tissues. However, most hydrogels are highly permeable, and without specific functionalization, protein drugs are rapidly released from the hydrogel matrix. Therefore, the success of using hydrogels to achieve pulsatile protein release control relies on two critical issues. One is how to prevent a rapid protein release from the hydrogels between protein delivery pulses; the other is how to induce protein release in a pulsatile manner when necessary. The first issue can be addressed if the hydrogel is functionalized to possess strong physical interactions with the protein drugs. For instance, a variety of promising hydrogels bearing peptides, heparin, or Ni²⁺ have been shown to absorb positively charged proteins or histidine-tagged proteins and to prevent their rapid release. However, there has not been a study to explore the feasibility of using these hydrogel systems for pulsatile release control, because there are no molecular triggers to induce the dissociation of those molecular pairs.

A novel particle-hydrogel composite was studied to address the aforementioned two critical issues by using nucleic acid aptamers and an intermolecular hybridzation mechanism. The novel composite was synthesized using affinity particles and hydrogels. The affinity particles were functionalized with nucleic acid aptamers on their surface. Nucleic acid aptamers are single-stranded oligonucleotides that are screened from DNA/RNA libraries using the systematic evolution of ligands by exponential enrichment.

Experiments were performed using a DNA aptamer and agarose hydrogel as a model. The results demonstrated that COs could penetrate the composite, hybridize with the aptamers tethered on the surface of the particles, and trigger a pulsatile protein release at different time points.

### Materials

### 1) Reagents

Streptavidin-coated polystyrene microparticles (1.3 μm) were purchased from Spherotech (Lake Forest, Ill.). N-ethyl-N-(3-diethylaminopropyl)carbodiimide (EDC), N-hydroxysuccinimide (NHS), phosphate buffer saline (PBS), agarose, ammonium persulfate (APS), N,N,N′,N′-tetramethylenediamine (TEMED), and a premixed solution of acrylamide and bis-acrylamide (40%; 29:1) were purchased from Fisher Scientific (Suwanee, Ga.). The anti-PDGF-BB aptamer (5′-NH2-AAA AAA AAA AAC AGG CTA CGG CAC GTA GAG CAT CAC CAT GAT CCT GTG ACT TGA GCA AAA T-3′, M_{W}=19 kDa; SEQ ID NO:3), the scrambled control aptamer (5′-NH2-AAA AAA AAA AAA ATG CCA CCT CGG TAG TCC TAA AGG GCA AAT TCG GAA CGC AGG TAC TTA C-3′, M_{W}=19 kDa; SEQ ID NO:4), the 31-nt CO (5′-ATT TTG CTC AAG TCA CAG GAT CAT GGT GAT G-3′, M_{W}=9.5 kDa; SEQ ID NO:5), and the scrambled CO (5′-TAG CCT GTG GAG TAT CGC TAA TCA GGC GGA T-3′, M_{W}=9.5 kDa; SEQ ID NO:6) were purchased from Integrated DNA Technologies (Coralville, Iowa). Recombinant human PDGF-BB (M_{w}=25 kDa) and bovine serum albumin (BSA) were purchased from R&D Systems (Minneapolis, Minn.) and Invitrogen (Carlsbad, Calif.), respectively. Biotinyl-N-hydroxysuccinimide (NHS-biotin) and 2-(N-morpholino) ethanesulfonic acid (MES) were purchased from Sigma-Aldrich (St. Louis, Mo.). 5K membrane filter unit was purchased from Millipore (Billerica, Mass.).

### 2) Prediction of Secondary Structures

The secondary structures of the aptamer, the CO and their hybridization format were generated by using the program RNAstructure version 4.6. The predicted structures with the lowest free energies were presented.

### 3) Preparation of Affinity Particles

The particle model was synthesized with streptavidin-coated polystyrene particles and biotinylated aptamers because the strong streptavidin-biotin interaction is virtually irreversible. The aptamer biotinylation was carried out by the reaction of NHS-biotin and primary amino groups at pH 7.0. The reaction mixture was then filtered through a 5K membrane filter unit to remove any free biotin molecules. 2.5 nmole of the aptamer and 1 mg of streptavidin-coated microparticles were added into 100 μL PBS and incubated for 30 minutes. The microparticles were then washed with PBS four times. The number of aptamers tethered to the particles was quantified from the amount of the aptamers found in the washing buffer using UV-vis spectrophotometer (ND-1000, NanoDrop Products).

### 4) Preparation of Affinity Composites

The 120 μg aptamer-functionalized particles were mixed with 4 ng PDGF-BB in 20 μL PBS for 30 minutes, using a 125:1 molar ratio of aptamer to PDGF-BB. The suspension was then added to 180 μL of 0.5% agarose solution at 40° C. and gently mixed. Finally, the particle-agarose suspension was cast into a cylindrical mold to form the composite at room temperature. The composite was cut into small volumes (50 μL each) for the protein release tests. The binding efficiency of PDGF-BB to the affinity particles was also quantified. The proteins and the particles were prepared with the same procedure as mentioned above. After 30 minutes of incubation, 30 μL of the suspension were well mixed with releasing media (the mixture of PBS, 0.1% BSA, 0.05% Tween-20, and 0.05% NaN₃) to a final volume of 1000 μL. The suspension was centrifuged at 10,000 rpm to separate the particles from the release media. Finally, the amount of unbound PDGF-BB in the release media was quantified by using a Human PDGF-BB ELISA kit (PeproTech, Rocky Hill, N.J.).

### 5) Rheology Characterization

The storage and loss moduli of the composite were measured with an AR-G2 rheometer (TA Instruments, New Castle, Del.). The composite samples were prepared as circular discs with a 20 mm diameter and a 1.5 mm thickness. The temperature was set at 37° C. for all experiments. A stress-sweep test was performed to confirm that the measurement was in the linear viscoelastic regime. The oscillation stress was varied from 0.01 to 1000 Pa at a fixed frequency of 1 rad/s and the frequency was varied from 0.1 to 100 rad/s at an oscillation stress of 1 Pa oscillation stress.

### 6) Gel Electrophoresis

The hybridization of the nucleic aptamer was examined by gel electrophoresis. The aptamer was mixed with either the CO or the scrambled CO (S-CO) at a molar ratio of 1:5 in 10 μL of PBS. The mixture was incubated at room temperature for 10 minutes and transferred into a 10% native polyacrylamide gel. The gel was subjected to electrophoresis with a Bio-Rad Mini-PROTEAN tetra cell and stained with ethidium bromide. The stained gel was imaged using a Bio-Rad GelDoc XR system (Hercules, Calif.).

### 7) Flow Cytometry

Approximately 2×10⁷ aptamer-functionalized particles were incubated in 100 nM of either scrambled CO or CO labeled with 6-carboxy-fluorescein (denoted as FAM) for 1 hour. The particles was centrifuged, washed once with 500 μL of PBS, and analyzed by a BD FACSCalibur™ flow cytometer (San Jose, Calif.).

### 8) Microscopic Examination

To examine the particles in an aqueous solution, the particle suspension was spread on the surface of a glass slide, covered by a cover slip, and subjected to a microscopic examination. To examine the particles and the intermolecular hybridization in the composite, the composite was incubated with FAM-labeled COs (100 nM) for 1 hour, washed with PBS three times, and finally incubated in PBS for 1 hour to remove free COs. The treated composite was manually cut to obtain a thin section (˜0.5 mm) and placed on a cover slip for a microscopic examination. Both an inverted microscope (Axiovert 40CFL, Carl Zeiss) and a Leica SP2 spectral confocal microscope were used in this study. For the inverted microscopy imaging, the images were processed by Q-Capture Pro 6.0 software. For the confocal microscopy imaging, the images were processed using the supplied Leica Confocal software.

### 9) Surface Plasmon Resonance (SPR) Analysis

The molecular interaction between the PDGF-BB and its aptamer was studied using the SPR spectrometry (SR7000DC; Reichert Analytical Instrument; Depew, N.Y.). A carboxyl group-functionalized sensor chip (Reichert Analytical Instrument; Depew, N.Y.) was activated with NHS and EDC for PDGF-BB immobilization. The binding solution of 100 nM of either anti-PDGF-BB aptamers or scrambled aptamers was flowed over the biochip for 5 minutes (30 μL/min) for the analysis of molecular association. Subsequently, the washing solution (PBS, PBS containing 500 nM of CO, or PBS containing 500 nM of S—CO) was introduced over the biochip for another five minutes (30 μL/min) for the analysis of molecular dissociation. The biochip was regenerated by flowing 1 M of NaCl in the channel for two minutes (100 μL/min) followed by the PBS. The dissociation rate constant (k_{off}) was obtained by fitting the binding profiles with the Scrubber 2.0 software as provided by the manufacturer.

### 10) Examination of PDGF-BB Release

The composites were incubated in 250 μl of releasing medium at 37° C. with a shaking rate of 60 rpm. At predetermined time points, the release medium was totally collected and a fresh medium was added. For the pulsatile release tests, 10 μL of 25 μM CO was added into the release medium to a final concentration of 100 nM at Days 5 and 15. After a 1-hour incubation period, the supernatant was collected and replaced with fresh release medium. At the end of the release experiments, the native agarose gels were minced for PDGF-BB elution to calculate the total amount of released PDGF-BB. PDGF-BB was quantified by using a human PDGF-BB ELISA kit. All experiments were performed in duplicate.

### Results

FIG. 8A schematically shows the overall procedure for the preparation of the composite. The aptamer model was a DNA oligonucleotide that could bind platelet-derived growth factor-BB (PDGF-BB) with high affinity. The particle model was synthesized with streptavidin-coated polystyrene particles and the biotinylated aptamers because the streptavidin-biotin interaction is extremely strong with a K_{d} of 4×10⁻¹⁴ M. To sufficiently adsorb PGDF-BB onto the particle surface, the affinity particles were incubated in PDGF-BB solution for 30 minutes before the whole mixture was transferred into an agarose solution. After gentle shaking, the suspension was cast into a cylindrical mold where the particle-hydrogel composite formed. Based on the particle information provided by the supplier, each particle could bind approximately 205,000 molecules of FITC-biotin, which is proportional to streptavidin binding sites. The results showed that 56,000 aptamers were successfully immobilized onto each particle. After mixing the affinity particles with PDGF-BB, it was found that the affinity particles could entrap the proteins with the efficiency of 93±2%. Because particle aggregation can affect the properties of a composite, the morphology of particles was characterized after each step. The microscopic examination showed that the adsorption of the aptamers or the proteins to the particle surface did not cause any particle aggregation (FIG. 8B). The examination of composite morphology also showed that the affinity particles were well distributed in the agarose hydrogel (FIG. 8C). The storage and loss moduli of each composite were also characterized (FIG. 8D) because the mechanical properties of drug delivery systems can affect their therapeutic efficacy and safety in real applications. The rheology data showed that the storage and loss moduli of the hydrogel and the composite were identical.

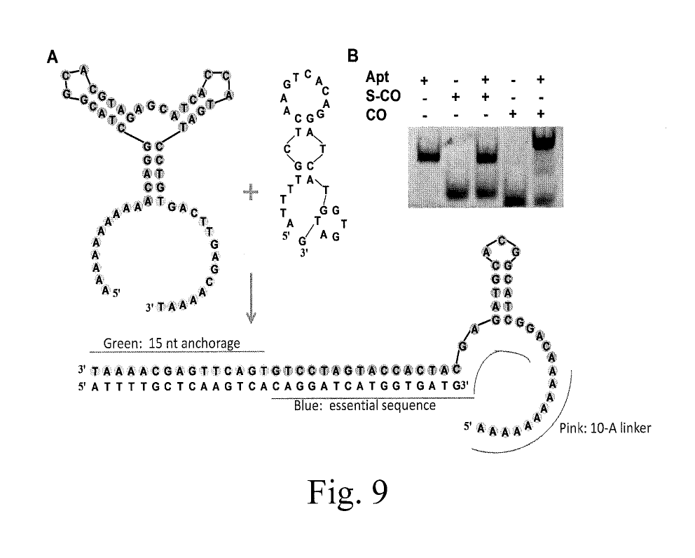

The novelty of this study lies not only in the incorporation of affinity particles into a hydrogel, but also in the control of particle functionality through intermolecular hybridization. Therefore, an aptamer and CO pair that could hybridize at the 3′ region of the anti-PDGF-BB aptamer was designed (FIG. 9A). The aptamer has three segments: a 10-A segment at the 5′ end, a 36-nucleotide (nt) segment in the middle, and a 15-nt segment at the 3′ end. The 10-A segment was used as a linker to tether the aptamers onto the particle surface and to minimize the steric hindrance in molecular recognition. The 36-nt sequence contained the essential nucleotides of the parent aptamer that was originally selected from a DNA library. This was used to recognize and bind PDGF-BB to the particle surface. The 15-nt segment at the 3′ end functioned as a molecular anchorage to facilitate the intermolecular hybridization between the aptamer and its CO. As the structure of an aptamer plays a critical role in determining its binding functionality, it is important that the 15-nt segment does not form an intramolecular hybridization structure with the essential 36-nt sequence. Otherwise, the binding strength of the aptamer to the target can decrease. The structural prediction showed that the 15-nt segment would not hybridize with the 36-nt sequence (FIG. 9A), indicating that the designed 61-nt aptamer could bind to PDGF-BB. On the other hand, it is necessary to induce a structural change through intermolecular hybridization once the aptamer interacts with the CO. This would inactivate the aptamer and induce the rapid release of the bound protein. As expected, the structural prediction showed that the CO could induce the structural change of the aptamer after the intermolecular hybridization (FIG. 9A).

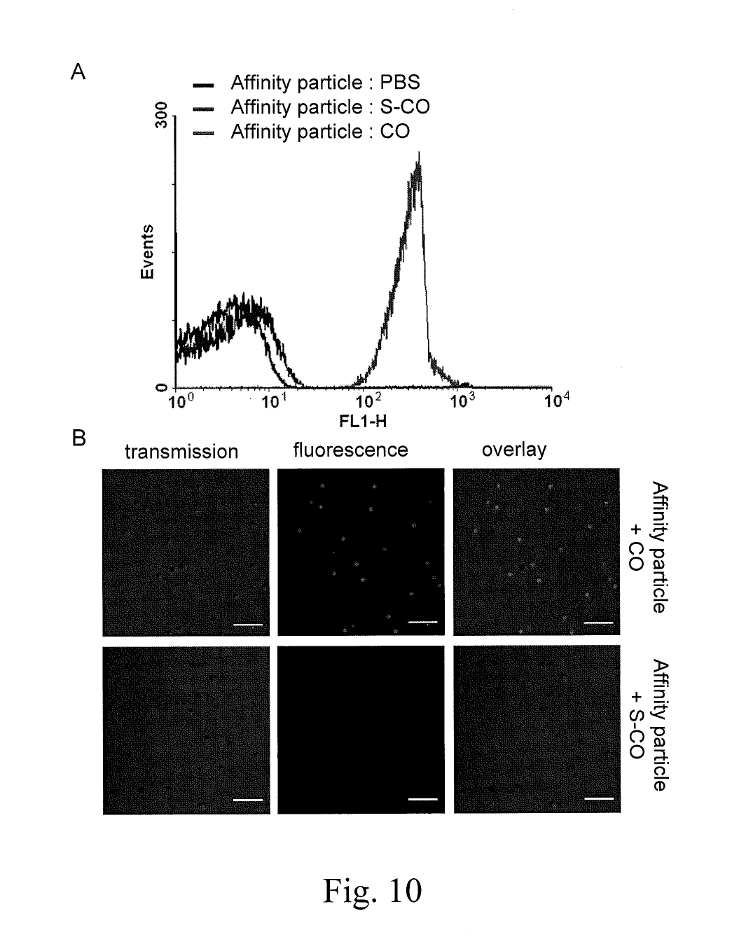

Besides the structural analyses, experiments were performed to directly illustrate whether the rational molecular design could enable effective intermolecular hybridization and accelerate aptamer-protein dissociation. Gel electrophoresis showed that the free aptamer and the CO could hybridize in an aqueous solution (FIG. 9B). To ensure that intermolecular hybridization could also occur on the particle surface, both flow cytometry analysis and microscopy imaging were used to examine the properties of the affinity particles. As shown in the histogram (FIG. 10A), the affinity particles became fluorescent after the treatment with FAM-labeled COs. The affinity particles were also treated with FAM-labeled scrambled COs to discern if the fluorescence signal was due to the specific or to the non-specific CO adsorption. Those particles in the control group did not exhibit a strong fluorescence (FIG. 10A). The confocal microscopy images (FIG. 10B) were consistent with the flow cytometry analysis. Clearly, these results show that the CO hybridized with the aptamer on the particle surface.

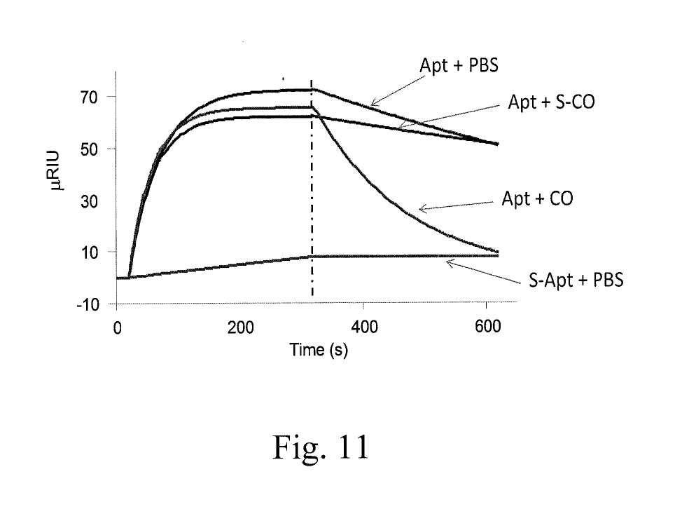

Because real applications were involve not only aptamers and COs, but also target proteins, surface plasmon resonance (SPR) was used to directly examine the intermolecular hybridization-mediated protein dissociation (FIG. 11). In the presence of scrambled COs, the dissociation rate constant (k_{off}) was 6.5×10⁻⁴ s⁻¹. In contrast, the k_{off} value was increased to 6.6×10⁻³ s⁻¹ when the functional COs were introduced to the system. Taken together, the SPR analysis indicated that the molecular pair of anti-PDGF-BB aptamer and its CO could be used for modulating the interactions between PDGF-BB and its aptamer.

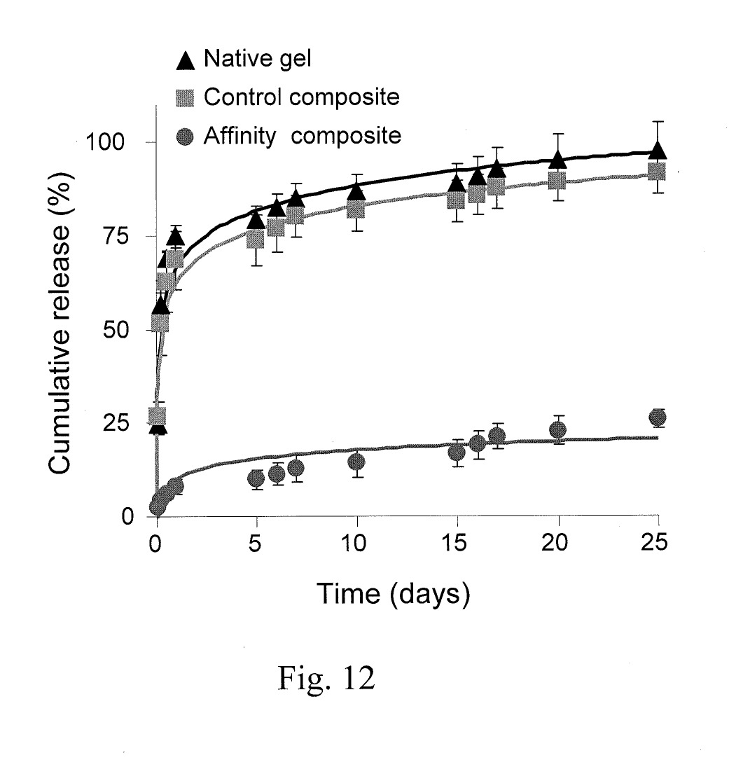

After studying the properties of affinity particles and the functionality of the molecular pair, particle-hydrogel composites for a controlled-release test were synthesized. Two sets of controlled-release experiments were carried out. The first experiment was aimed to address the first aforementioned question, i.e., whether affinity particles would prevent the rapid release of the target proteins from the composite. As shown in FIG. 12, during the first 24 hours, ˜70% of the PDGF-BB molecules were released from both the native agarose gel and the control composite. The significant burst release clearly demonstrated that the native hydrogel or the control composite could not prevent a rapid protein release. In contrast, the initial burst release was dramatically reduced to ˜8% for the functionalized composite. This burst release was presumably due to the existence of free PDGF-BB molecules that were initially not bound to the affinity particles during the composite preparation. After the initial burst release, the PDGF-BB release from the functional composite was very slow. The average daily release rate was approximately 0.75% between Day 2 and Day 25. Clearly, the results show that the affinity particles could efficiently prevent rapid PDGF-BB release from the functionalized composite.

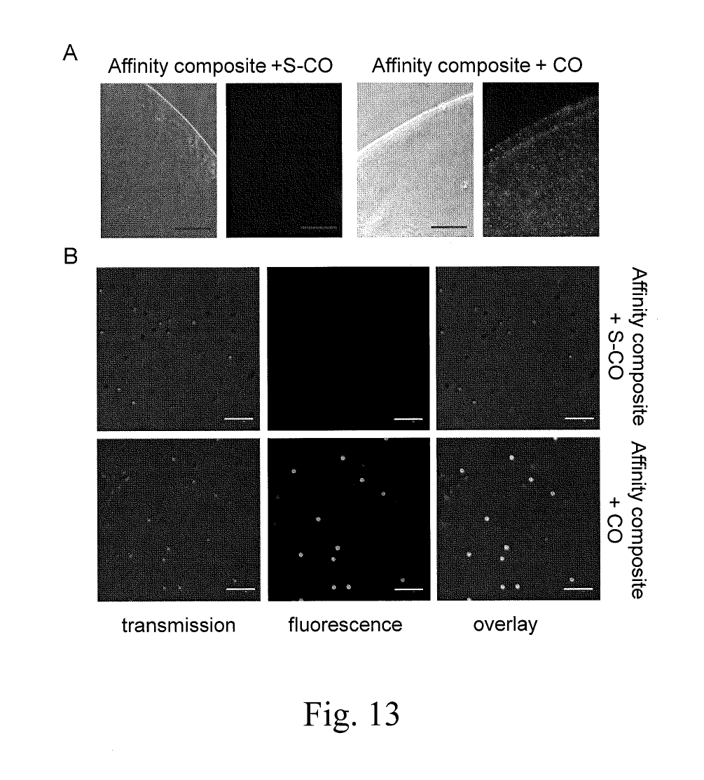

The second controlled-release experiment was aimed to address the question of whether intermolecular hybridization would induce a protein release in a pulsatile manner. Experimentally, the capability of the COs in hybridizing with the affinity particles in the composite was first characterized. Similar to the observation in the aqueous solution (FIG. 10B), the affinity particles in the composite became fluorescent after treating the composite with FAM-labeled COs (FIGS. 13A & B). This result showed that the COs could easily penetrate the composite and hybridize with the affinity particles in the composite environment.

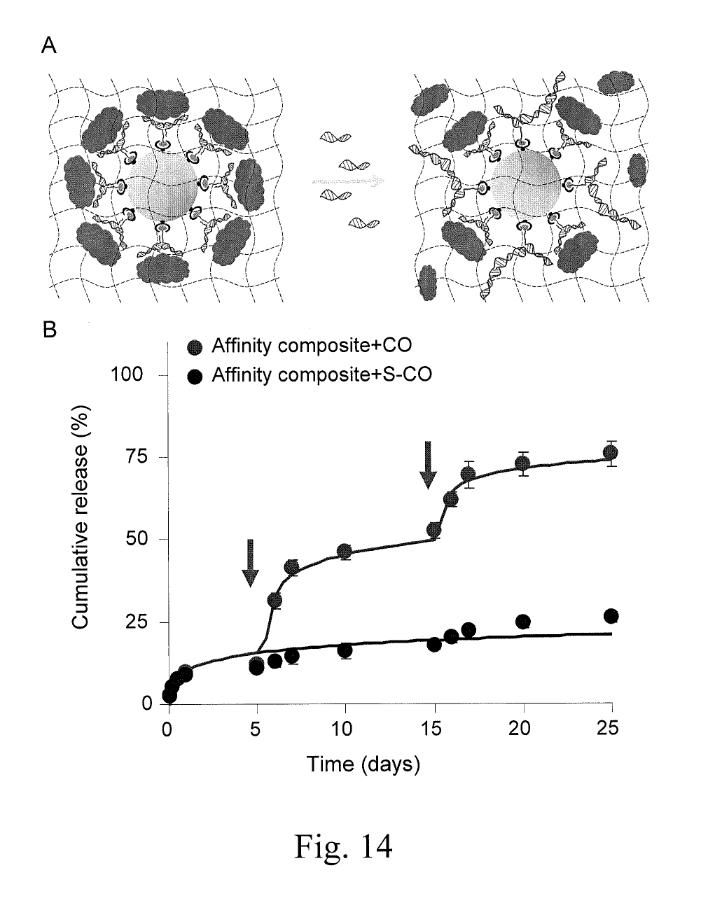

The protein release from particle surface or the functional composite is schematically illustrated in FIG. 14A. The pulsatile protein release patterns are shown in FIG. 14B. The one-hour stimulation of the composite with the COs led to the pulsatile PDGF-BB release. The two pulse release rates were ˜20% and ˜10% per day, respectively. Clearly, these data show that the composite could release PDGF-BB in a pulsatile manner through the intermolecular hybridization mechanism.

Previously, various promising drug delivery systems have been developed to improve pulsatile protein release control. Stimuli such as temperature, light, electric potential, mechanical force, enzymes, calcium, drugs, and antigens have been applied to induce the structural changes of hydrogels (e.g., decomposition or deformation) for protein release. Different from these elegant protein release systems, the protein release system demonstrated in this study is based on aptamer-protein association and CO-mediated aptamer-protein dissociation. It has the following special characteristics.

First of all, because the release of proteins is governed by the aptamer-protein interactions and the intermolecular hybridization, the protein release does not rely on the structural change of the composite. Thus, it is possible to achieve a long-term protein release control at predetermined multiple time points. Second, recent progress in nucleic acid research has shown that oligonucleotides can be chemically functionalized (e.g., PEGylation) to acquire desired in vivo half-life and good biocompatibility. Thus, this system holds great potential for use in vivo. For instance, angiogenesis is a complicated procedure, involving multiple growth factors. This novel composite can be used for incorporating multiple aptamers and growth factors and then be implanted in vivo for sequential release of multiple growth factors. At predetermined time points, complementary oligonucleotides can be systemically administered. After reaching and diffusing into the implant with the aid of blood stream, complementary oligonucleotides can trigger the in situ pulsatile release of the incorporated growth factors. Third, in principle, aptamers can be selected to strongly and specifically bind any molecule of interest ranging from large proteins to small molecules. Thus, the same concept can be applicable to the delivery of not only protein drugs but also non-protein drugs. Fourth, the synthesis of the composite does not have to require any specific interactions or chemical crosslinking between the particles and the hydrogel. Therefore, it is possible that any type of biocompatible hydrogel can be utilized to develop a particle-hydrogel composite for pulsatile protein release control.

### Conclusion

This study shows that the novel particle-hydrogel composite is a promising system for pulsatile protein release control. The composite can effectively prevent the rapid release of proteins in normal conditions because the proteins can be strongly adsorbed on the surface of affinity particles through the strong aptamer-protein interactions. Moreover, the composite can release proteins in a pulsatile manner after it is stimulated by small complementary oligonucleotides to induce the protein-aptamer dissociation. It is believed that this pulsatile protein delivery system holds great potential for various biological and biomedical applications such as tissue engineering and regenerative medicine.

### Example 6

### An Aptamer-Functionalized In Situ Injectable Hydrogel for Controlled Protein Release

Hydrogels are made of hydrophilic polymers that can be either natural biomolecules or synthetic materials. The hydrogel networks are usually formed by the crosslinking of polymer chains via covalent bonds, hydrogen bonds, or ionic interactions. Hydrogels can be synthesized outside of the body for in vivo implantation. Alternatively, a polymer solution can be rationally designed and directly injected into the desired site where the solution is transformed into a hydrogel (i.e., in situ gelation). In comparison to hydrogel implants, the in situ gelation of polymer solutions has special merits for in vivo applications. An injectable hydrogel can be delivered in vivo with minimal surgery and, in principle, can fill cavities with any geometry. Thus, the delivery of in situ injectable hydrogel formulations would not only improve patient compliance and quality of life, but also avoid the need of fabricating patient-specific hydrogel implants. The in situ gelation can be simply achieved through diverse mechanisms, such as temperature-induced phase transition, UV-mediated polymerization, polyelectrolyte complexation, solvent exchange, and self-assembly. Because of these merits, the in situ injectable hydrogels have been widely studied for protein delivery. However, most hydrogels are highly permeable, which can lead to the rapid release of loaded proteins. Thus, there is a clear need to develop novel methods for improving the properties of injectable hydrogels to achieve desired protein release kinetics.

In this example, various methods for synthesizing in situ injectable hydrogels to control the release of proteins by using nucleic acid aptamers as affinity sites of the proteins in the hydrogel are described. The model aptamer used in this study was previously selected against platelet-derived growth factor B (PDGF-B) from a DNA library by using a gel electrophoresis-based SELEX approach. Further, to understand the effect of sequence modifications on the binding functionality of this model aptamer, a series of anti-PDGF aptamers were generated either by randomizing the nonessential nucleotide tail or by mutating the essential nucleotides. The functionality of these aptamers was studied by the examination of their secondary structures and dissociation constants. Based on these studies, several aptamer sequences were selected to investigate the protein release from an aptamer-functionalized in situ injectable poloxamer hydrogel. Further, poloxamer is a block copolymer that has been proposed for a variety of pharmaceutical applications, such as the delivery of growth factors and viruses. The results demonstrated that the aptamer-functionalized poloxamer hydrogels could slowly release PDGF with tunable kinetics.

### Materials

All the DNA molecules were purchased from Integrated DNA Technologies (Coralville, Iowa) and listed in Table 1. Recombinant human PDGF-BB and bovine serum albumin (BSA) were purchased from R & D Systems (Minneapolis, Minn.) and Invitrogen (Carlsbad, Calif.), respectively. Poloxamer 407 (Pluronic F-127), biotinyl-N-hydroxysuccinimide (NHS-biotin), and 2-(N-morpholino) ethanesulfonic acid (MES) were purchased from Sigma-Aldrich (St. Louis, Mo.). The 5K membrane filter unit was purchased from Millipore (Billerica, Mass.). Streptavidin-coated microparticles (1.3 were purchased from Spherotech (Lake Forest, Ill.). N-ethyl-N-(3-diethylaminopropyl) carbodiimide (EDC), N-hydroxysuccinimide (NHS), and phosphate buffered saline (PBS), disodium hydrogen phosphate (Na₂HPO₄), Tween 20, and sodium azide (NaN₃) were purchased from Fisher Scientific (Suwanee, Ga.).

TABLE 1
List of DNA sequences.

| | | SEQ |
|---|---|---|
| | | ID |
| | Nucleotide sequence (5′→3′) | NO: |
| S1 | GCGATACTCC ACAGGCTACGGCACGTAGAGCATCACCATG | 7 |
| | ATCCTG | |
| S2 | CAATTCCGCG ACAGGCTACGGCACGTAGAGCATCACCATG | 8 |
| | ATCCTG | |
| S3 | CCACGGTCTA ACAGGCTACGGCACGTAGAGCATCACCATG | 9 |
| | ATCCTG | |
| S4 | CGCCATTCAG ACAGGCTACGGCACGTAGAGCATCACCATG | 10 |
| | ATCCTG | |
| S5 | CGCATGCTCA ACAGGCTACGGCACGTAGAGCATCACCATG | 11 |
| | ATCCTG | |
| S6 | TCGCACATGC ACAGGCTACGGCACGTAGAGCATCACCATG | 12 |
| | ATCCTG | |
| S7 | GCCGTTCCAA ACAGGCTACGGCACGTAGAGCATCACCATG | 13 |
| | ATCCTG | |
| S8 | TGCCATGCCA ACAGGCTACGGCACGTAGAGCATCACCATG | 14 |
| | ATCCTG | |
| S9 | GCAACTGCTC ACAGGCTACGGCACGTAGAGCATCACCATG | 15 |
| | ATCCTG | |
| S10 | CATGAGCCCT ACAGGCTACGGCACGTAGAGCATCACCATG | 16 |
| | ATCCTG | |
| M1 | GCGATACTCC ACAGGCTACGGCACGTAGAGCATCACCATG | 17 |
| | ATCCT [symbol] | |
| M2 | GCGATACTCC ACAGGCTACGGCACGTAGAGCATCACCATG | 18 |
| | ATCC [symbol] | |
| M3 | GCGATACTCC ACAGGCTACGGCACGTAGAGCATCACCATG | 19 |
| | ATC [symbol] | |
| S-S1 | GCGATACTCC ATCAATGGACCGCGCACTCGCCAGTGCTAA | 20 |
| | TGGCAA | |
| FAM- | FAM-CAGGATCATGGTGATGCT CTACGTGCCGTA | 21 |
| CO | | |

Note:
The nonessential nucleotide tail is underlined. The mutated nucleotides are italic and bold. CO: complementary oligonucleotide.

### 1) Modification of Aptamer Sequence

The sequence of the aptamer was modified through either tail variation or stem mutation. For the tail variation, the tail was generated by sequence randomization with A:C:G:T ratio maintained the same. For the stem mutation, the first three nucleotides G, T, and C at 3′-end of the aptamer S1 were replaced with A, C, and T, respectively. The aptamers with one, two and three mutation sites were denoted as M1, M2, and M3, respectively. The sequences of modified aptamers are shown in Table 1.

### 2) Structure Prediction

The secondary structures of these aptamer sequences were predicted by using the program RNAstructure version 4.6. This program were used to generated the secondary structures of both RNA and DNA oligonucleotides. The secondary structures with the lowest free energies were used for presentation and analysis.

### 3) Measurement of Binding Affinity

The affinities of the aptamers were measured with surface plasmon resonance (SPR) spectrometry (SR7000DC, Reichert Analytical Instrument, Depew, N.Y.). Carboxyl group-functionalized sensor chips were purchased from the Reichert Analytical Instrument. The chips were initially activated with 0.2 M EDC/0.1 M NHS for 10 min After the activation step, PDGF-BB (20 μg/ml in 25 mM Na₂HPO₄ at pH 8.5) was flowed over the activated sensor chip for protein immobilization. Before the test, the system was equilibrated with the running buffer for 30 min. The running buffer was made of PBS buffer (pH 7.4) containing 0.05% Tween 20. During the test, the aptamer solution was flowed over the sensor chip at a flow rate of 30 μL/minute for 5 minutes. Subsequently, the flowing solution was switched to the running buffer for molecular dissociation. After each test, the sensor chip was regenerated by flowing 1 M NaCl in the channel for 2 minutes and then washed with the running buffer. To determine the dissociation constants, a series of aptamer solutions were prepared with the concentration ranging from 3.13 to 200 nM. The data were processed with the Scrubber 2.0 software (BioLogic Software, Australia). The responses from the reference channel were subtracted from the responses from analyte channel before data analysis to minimize the effects of reflective index changes, nonspecific binding, and instrument drift. The dissociation constant (K_{D}) was determined by fitting the data to a simple biomolecular “1:1 binding” model.

### 4) Preparation of Aptamer-Functionalized Poloxamer Hydrogel

The aptamers with a primary amine group at the 5′-end were reacted with NHS-biotin at pH 7.0 overnight. The free biotin was removed from the reaction mixture by filtration through a 5K membrane filter unit. The aptamer solution containing a total of 2.5 nmol of aptamers was mixed with 1 mg of streptavidin-coated polystyrene particles in 100 μL PBS. After a 30-minute incubation, the functionalized particles were washed with PBS for four times. To prepare the affinity poloxamer hydrogel, 80 μg of aptamer-functionalized particles were first incubated with 4 ng of PDGF-BB in 20 μL PBS for 30 minutes at room temperature. The suspension was then mixed with 1,000 μL of 20% w/w poloxamer solution at 4° C. Finally, 250 μL of suspension was transfered into a 2 mL tube and allowed to form the hydrogel at 37° C. for 30 minutes before the hydrogel characterization and the release studies.

### 5) Flow Cytometry

Flow cytometry experiments were performed to examine the functionalization of the particles with the aptamers. Approximately 2×10⁷ particles were incubated in 100 nM of complementary oligonucleotide labeled with 6-carboxy-fluorescein (denoted as FAM-CO) for 1 hour. The particles were then washed twice with 500 μL of PBS before subjected to flow cytometry analysis with a BD FACSCalibur™ flow cytometer (San Jose, Calif.). A total of 10,000 events were collected for data analysis.

### 6) Microscopic Examination

The particle suspensions were spread on the surface of glass slides, covered with a cover slip, and examined by using a Leica SP2 spectral confocal microscope with a 100× objective. The images were processed using the Leica Confocal software provided by the manufacturer.

### 7) Rheology Characterization

The storage (G′) and loss (G″) moduli of the hydrogels were measured with an AR-G2 rheometer (TA Instruments, New Castle, Del.). Approximately 200 μL of cold particle-hydrogel suspension was loaded into the chamber. The experiments were performed with a oscillation mode. To ensure the validity of the data, a linear viscoelastic regime was first determined by performing a stress-sweep experiment at both 4 and 37° C. The oscillation stress was varied from 0.01 to 1,000 Pa at a fixed frequency of 0.1 Hz. The temperature-dependent moduli were measured from 4 to 45° C. with a fixed oscillation stress (6 Pa) and a constant heating rate (2° C/.min) The gelation point was defined as the crossover point of G′ and G″. In addition, the time-sweep modulus of the poloxamer solution with or without particles was measured at 37° C. for 1 hour. The oscillation stress was fixed at 6 Pa during the measurement.

### 8) Measurement of PDGF-BB Release

The release medium (i.e., PBS containing 0.1% BSA, 0.05% Tween-20, and 0.05% NaN₃) was first pre-warmed to 37° C. Next, 500 μL of release medium was carefully added into the tube containing 250 μL of poloxamer hydrogel. The tubes were incubated at 37° C. with a shaking rate of 60 rpm. At predetermined time points, 500 μL of release medium was carefully collected from the solution and replenished with 500 μL of fresh release medium. PDGF-BB in the release medium was quantified with a human PDGF-BB ELISA kit (PeproTech, Rocky Hill, N.J.). The experiments were performed in triplicate. The total amount of PDGF-BB in the native hydrogel was analyzed at the end of the release experiment to calculate the cumulative release of PDGF-BB.

### Results

The model aptamer can bind PDGF-BB with high affinity and specificity. Its original sequence contains 86 nucleotides. In general, approximately 10-15 nucleotides of an aptamer form a functional structure to interact with the target. These nucleotides exhibit the structures such as hairpin loops, quartet loops, bulges, or pseudoknots. However, these nucleotides require presentation in the context of the “parent” aptamer to achieve a sufficient binding capability. Thus, the essential nucleotides of an aptamer include not only the nucleotides forming a functional structure, but also those providing the right context to facilitate the formation of the functional structure. An aptamer usually contains 25-40 essential nucleotides. The other nucleotides are nonessential because they do not bind the target or facilitate the binding of the aptamer. The previous study has shown that the anti-PDGF-BB aptamer with 36 essential nucleotides can bind PDGF-BB with high affinity. One of the main purposes of this study is to understand the effect of sequence modifications on the binding functionality of the aptamer. Thus, two methods for tuning the binding functionality of the anti-PDGF-BB aptamer were used: (1) changing the context of the essential nucleotides by varying the composition of a nonessential nucleotide tail; and (2) mutating the stem of the aptamer.

### Effects of Tail Composition on Secondary Structure and Binding Functionality

Because the binding capability of a nucleic acid aptamer is dependent on its functional structure, the sequence and structure of the 36-nt aptamer were changed by attaching a nonessential nucleotide tail to its 5′-end. The tail contained 10 nucleotides. The hypothesis was that the nonessential nucleotide tail could form intramolecular base pairs with the essential nucleotides. As a result, the variation of the tail would change the context of the essential nucleotides and the binding affinity of the aptamer.

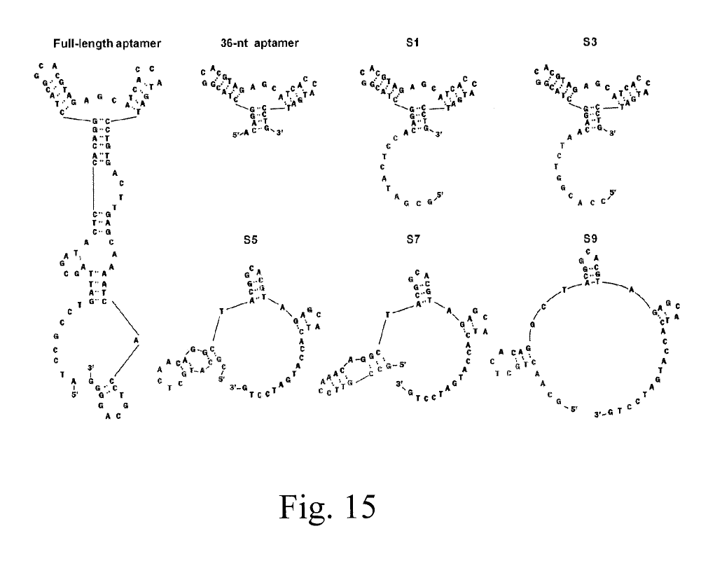

The hypothesis was first tested by analyzing the secondary structures predicted with the program RNAstructure. As shown in FIG. 15, the 36 essential nucleotides adopt a secondary structure which has three stem regions radiating from a common junction. The 36-nt aptamer and the 36 essential nucleotides in the aptamer S1 (i.e., the sequence truncated from the full-length aptamer without randomization; FIG. 15) virtually exhibit the same structure. The 5′- and 3′-ends form a four-base-pair stem.

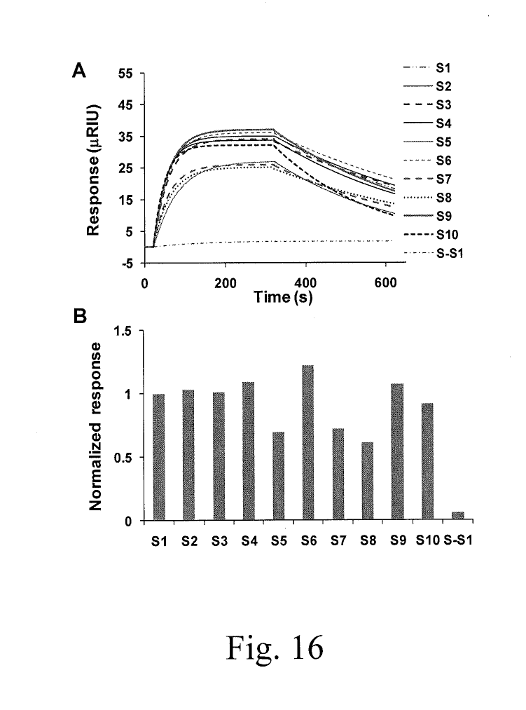

Ten aptamers with differential tail compositions and one scrambled aptamer were tested. The representative structures of the aptamers are shown in FIG. 15. The aptamer S1 was composed of 36 essential nucleotides and 10 nonessential nucleotides of the “parent” aptamer. The other nine aptamers was composed of 36 essential nucleotides and a randomized sequence of the 10-nt tail. The scrambled aptamer S-S1 was generated by randomizing the 36 essential nucleotides of aptamer S1. Two aptamers, S2 and S3, exhibited the same three-stem structure as the 36-nt aptamer. Others exhibited a completely different structure. Because the structure of an aptamer plays a critical role in determining its binding capability, the structural prediction indicated that aptamer S2 and aptamer S3 could bind PDGF-BB, whereas the others might not. However, as shown in FIG. 16, the results from SPR analysis were not in full agreement with the predictions of the secondary structures. Six aptamers (S2, S3, S4, S6, S9 and S10) virtually exhibited the same binding capability as the aptamer S1. The other three aptamers (S5, S7 and S8) exhibited weaker binding capability.

The difference between the structural predictions and the experimental measurements can result from different working conditions. Current algorithms (e.g., the RNAstructure program) were developed to predict the secondary structures of aptamers in a clean system. A clean system accounts for only the aptamers in a buffer solution. However, the SPR analysis is used to analyze the interactions of a molecular pair in a buffer solution. Thus, in addition to the aptamers, the system also has proteins. The proteins compete with the nucleotide tail in binding to the essential nucleotides. If the intermolecular interaction between the proteins and the essential nucleotides is much stronger than the intramolecular base paring between the nucleotide tail and the essential nucleotides, the tail may not interfere with the structural formation of the essential nucleotides in the presence of the target protein. In fact, as shown in the predicted structures, the intramolecular interactions did not result in a large number of Watson-Crick base pairs in some aptamers (e.g., S9), which indicates that the nucleotide tail interacts weakly with the essential nucleotides. Previous studies have shown that aptamers can undergo conformational changes upon binding to a target. In addition, the essential nucleotides of aptamers originally hybridized with shorter complementary oligonucleotides can change their structures in the presence of target molecules, and still bind their target. Thus, both the previous studies and this study indicate that the essential nucleotides may have the capability of forming the functional structure in the presence of their target molecules, and that the variation of a nonessential nucleotide tail, at least in the current system, may not significantly change the affinity of the aptamer.

### Effects of Stem Mutation on Secondary Structure and Binding Functionality

Because the randomization of a nonessential nucleotide tail did not result in significant interference with the aptamer-protein interactions, the essential nucleotides in the four-base-pair stem formed at the 5′- and 3′-ends were further mutated. The difference between these two methods is that the former one did not change the sequence of the essential nucleotides whereas the later one directly changed it.

Three aptamer mutants were generated: M1, M2, and M3. Their sequences are shown in Table 1.

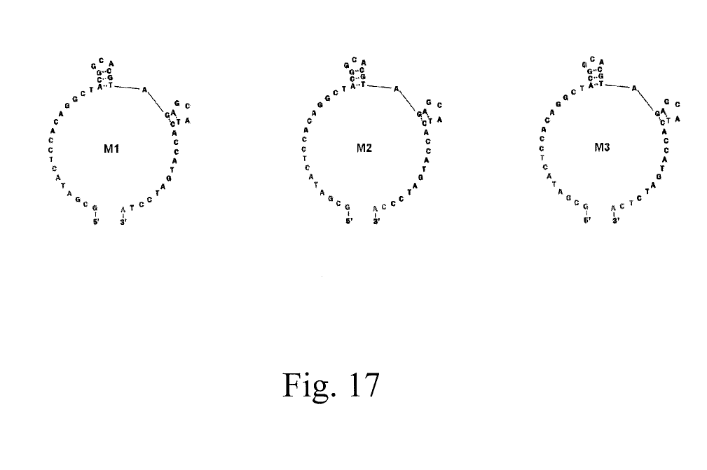

These mutants have one, two, or three mutated nucleotides at the 3′-end, respectively. These three aptamer mutants exhibit the same structural format as predicted by the program RNAstructure (FIG. 17). However, these secondary structures do not resemble that of aptamer S1 (FIG. 15). Aptamer S1 has a four-base-pair stem at the 5′- and 3′-ends whereas none of the mutants have a base pair at the 5′- and 3′-ends according to the structural prediction. Even aptamer M1 with only one mutated nucleotide does not form a stem at its 5′- and 3′-ends (FIG. 17).

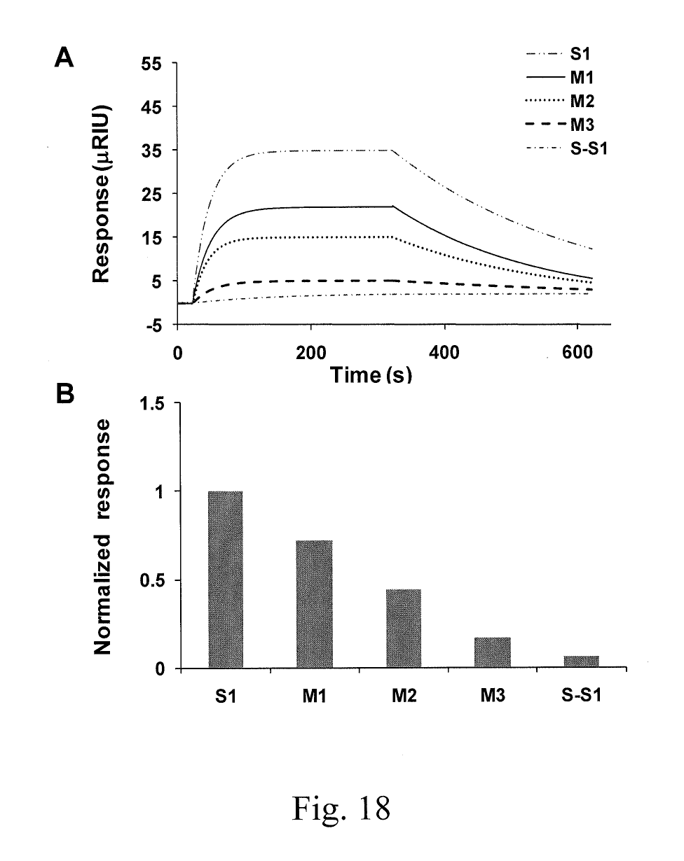

Interestingly, though the predicted stem-loop structures of the three mutants are the same, the SPR analysis showed that each exhibited a different capability of binding PDGF-BB (FIG. 18). The maximal SPR response decreased with an increasing number of mutations, indicating that the binding capability decreased. As discussed earlier, some of the essential nucleotides play a role of forming the functional structure for binding the target while the others provide a context to stabilize the structure. It is likely that the stem at the 5′- and 3′-ends of the sequence of the essential nucleotides plays a role of stabilizing the functional structure. When this region had mutations, the stem stability was decreased. Though the presence of the target protein might aid in facilitating the formation of the functional structure, it was not enough to overcome the instability resulting from the mutations in the stem region. As a result, the overall structural stability was decreased. The degree of decreased stability was dependent on the number of the mutations.

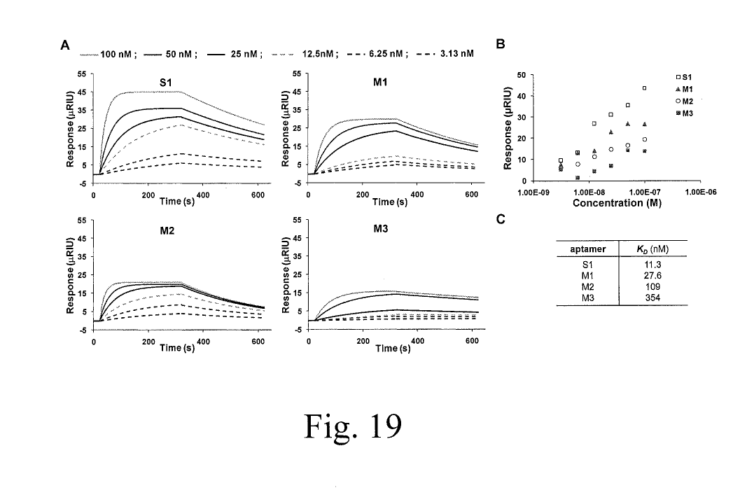

The interaction of a molecular pair was determined by not only molecular association but also molecular dissociation. The dissociation rate constant divided by the association rate constant is defined as the equilibrium dissociation constant (K_{D}). Thus, a series of aptamer solutions were prepared and the aptamer-protein interactions were characterized by calculating the dissociation constants (FIG. 19). The concentration-dependent responses were processed to calculate K_{D} using an equilibrium analysis. This analysis approach minimized the inaccuracy due to the limitation of mass transport on the biochip surface. The K_{D} values of M1, M2, and M3 were 27.6, 109, and 354 nM, respectively. These data clearly showed that the binding affinity of the aptamer decreased with the increasing number of mutations.

### Preparation and Characterization of Aptamer-Functionalized Poloxamer Hydrogel

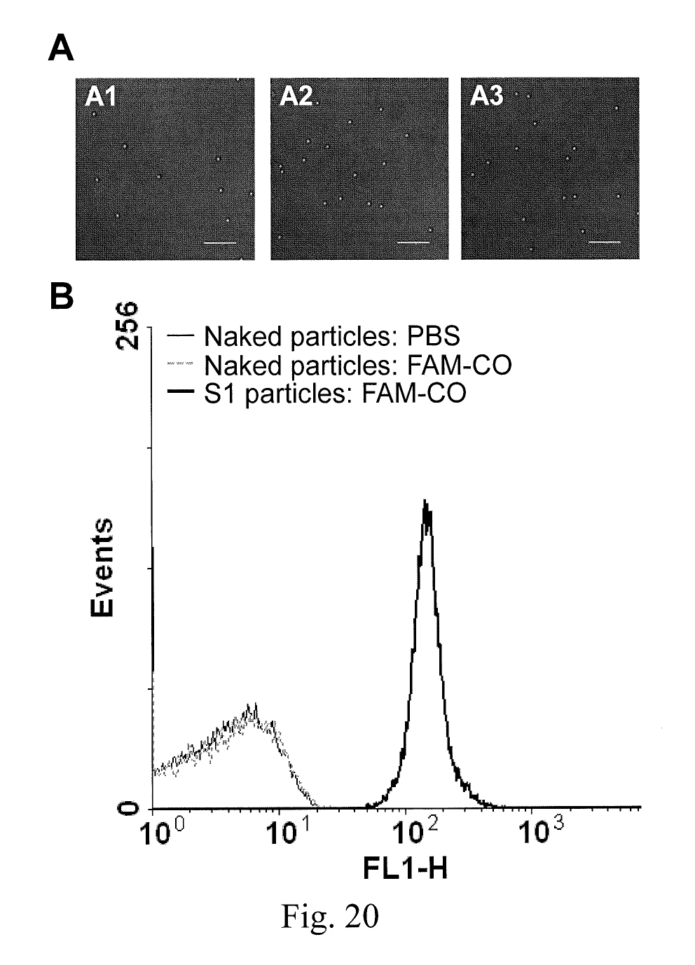

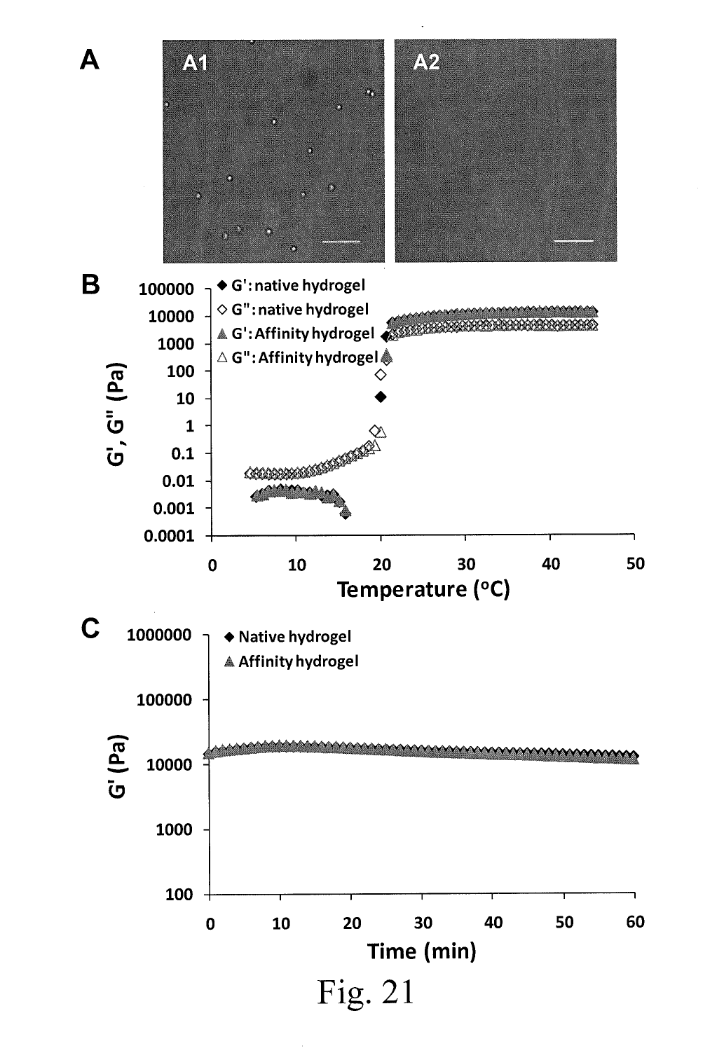

The affinity hydrogel was synthesized by mixing the aptamer-functionalized particles with a poloxamer solution (20% w/w). First, the properties of the particles were characterized by microscopy and flow cytometry (FIGS. 20A and 20B). The microscopic observation indicated that the overall particle morphology did not change after the functionalization with the aptamers or after the adsorption of PDGF-BB, and that there was no significant particle crosslinking or aggregation. To confirm that the anti-PDGF-BB aptamers were tethered to the particles, FAM-labeled complementary oligonucleotides were used to treat the particles. The flow cytometry data showed the presence of the complementary oligonucleotides on the particle surface, indicating that the aptamers were tethered to the particles. The properties of the affinity hydrogel with microscopic observation and rheology analysis were then characterized. The microscopy images showed that the particles were well distributed in the hydrogel (FIG. 21A). The rheology data demonstrated that the mechanical properties were not changed after the incorporation of the particles into the poloxamer hydrogel (FIG. 21B).

Affinity hydrogels are usually synthesized by the chemical conjugation of the ligands to the backbone of polymers. However, previous studies showed that a significant amount of affinity ligands could not be incorporated into the hydrogel network during the formation of hydrogels. This would negatively affect the loading efficiency of the proteins into the system. In addition, the free affinity ligands would diffuse out of the hydrogel, and could bind and inactivate protein drugs during the protein release. This problem can be significant if a higher concentration of affinity ligands is required. One may propose to wash free ligands out of the hydrogel. However, it is not possible to prewash hydrogels for in situ applications. This study shows a different approach for developing affinity hydrogels because the preparation of the hydrogels does not need any chemical conjugation between the aptamers and the hydrogel. This system only requires the physical mixing of the affinity particles, the protein drugs, and the polymer solution. Free aptamers can be removed before the particles are mixed with protein drugs and polymer solution. Thus, it is possible that any type of injectable hydrogel can be functionalized with nucleic acid aptamers by using this method for preparing an in situ injectable affinity hydrogel for controlled protein release.

### Protein Release From Aptamer-Functionalized Poloxamer Hydrogel

A variety of in situ injectable hydrogels have been studied for the delivery of protein drugs. One of them is thermo-sensitive hydrogels. Because their solutions can be easily transformed into a gel state at the body temperature, they have been widely used for protein delivery in the field of regenerative medicine and tissue engineering. In this study, poloxamer 407 was used as the model to investigate the capability of aptamers in controlling the protein release. Poloxamer 407 was a suitable model because it has been well-studied for drug delivery and its solution can be transformed into a gel state by increasing the temperature from a low degree to a high degree (e.g., body temperature). For instance, the 20% w/w solution formed a hydrogel with the temperature increased to approximately 20° C. as determined by the crossover point of G′ and G″ (FIG. 21B).

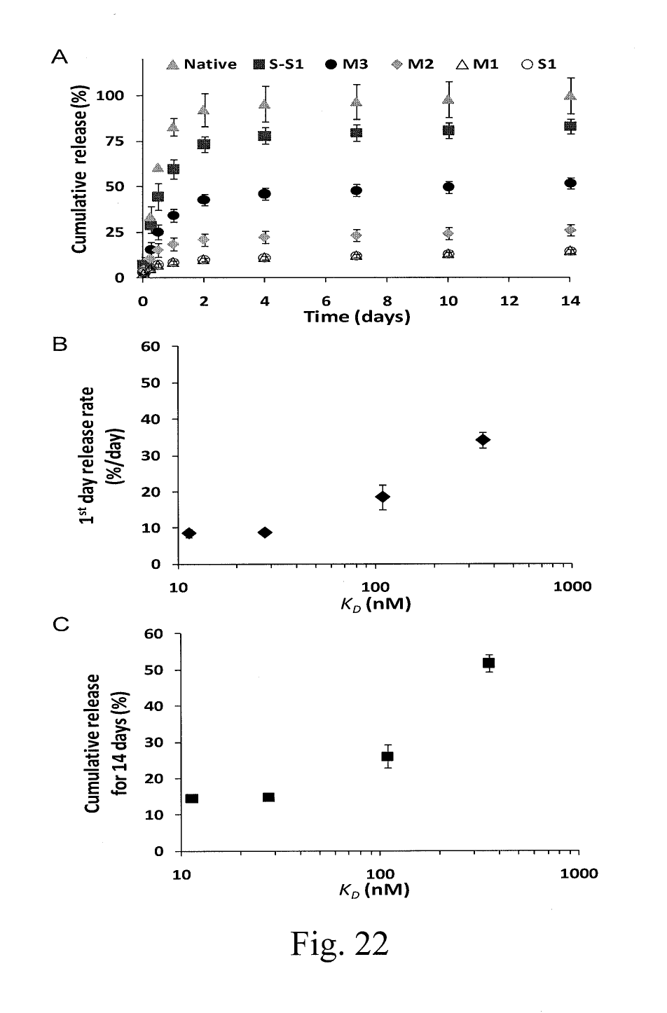

Drug release from a native hydrogel is governed by both drug diffusion and poloxamer dissolution. The poloxamer hydrogel was directly incubated in the release medium with no membrane to separate the hydrogel from the release medium. Thus, water uptake, poloxamer dissolution, and protein release could happen simultaneously. When the poloxamer hydrogel starts to dissolve in the release medium, its hydrogel concentration decreases. Because the formation of a poloxamer hydrogel is not only a function of temperature, but of concentration as well, the decrease of the concentration of the hydrogel will in turn accelerate its dissolution. The drug release rate will increase in parallel. Previous studies have shown that the drug release rate and poloxamer dissolution follow zero-order kinetics. The data also showed that PDGF-BB release from a native poloxamer hydrogel (i.e., poloxamer hydrogel without aptamer-coated particles) was fast and exhibited apparent zero-order kinetics during the first day (FIG. 22). More than 80% of the loaded PDGF-BB was released during the first day. The fast release will not only raise the cost of treatments, but will also lead to a wide distribution of protein drugs in non-target tissues and cause side-effects in vivo. This is a particularly important issue in the delivery of a protein drug (e.g., interleukin 2) with a narrow therapeutic index.

In contrast, the PDGF-BB release from the aptamer-functionalized poloxamer hydrogels (i.e., poloxamer hydrogels with aptamer-coated particles) was significantly decreased (FIG. 22). For instance, less than 10% of the loaded PDGF-BB was released from the S1-functionalized hydrogel during the first day. A total of 14.5% was released within the first two weeks. The strong molecular interaction between proteins and aptamers creates a significant barrier for protein diffusion and therefore retard the release of proteins from the hydrogels. The results also showed that the release rates of PDGF-BB could be controlled by varying the affinity of the anti-PDGF-BB aptamer (FIG. 22). The first-day release rate and the cumulative release at the end of the experiment decreased with the decrease of the K_{D} value (FIGS. 22B and 22C). The higher affinity indicates the stronger molecular interaction. The stronger molecular interaction retards the diffusion of proteins from the particle surface and the hydrogel more significantly. Thus, the release was slower when poloxamer hydrogels were functionalized with higher affinity aptamers.

### Conclusion

Structural predictions and binding analysis were used to study a number of anti-PDGF-BB aptamers that were generated by the mutation of the essential nucleotides and the variation of the nonessential nucleotides. The results showed that the mutation of the essential nucleotides in the stem region significantly altered the binding affinity of the aptamer, depending on the number of mutations. In contrast, the sequence modifications of the nonessential nucleotide tail did not significantly alter the affinity of the aptamer. The difference between the structural predictions and the experimental measurements indicated that the aptamers could undergo structural changes in the presence of their target molecules. The aptamers were further used to functionalize the poloxamer hydrogel. The functionalization did not need any specific chemical modification of the hydrogel. The release tests demonstrated that PDGF-BB was rapidly released from the native poloxamer hydrogel. In contrast, its release from the aptamer-functionalized hydrogels was significantly prolonged. The release rate could be controlled by adjusting the affinity of the aptamer. Therefore, the results demonstrate that nucleic acid aptamers, in principle, can be applied to functionalize any in situ injectable hydrogel for controlled protein release.

### Example 8

### Preparation of Affinity Nanogel for Controlling Protein Release

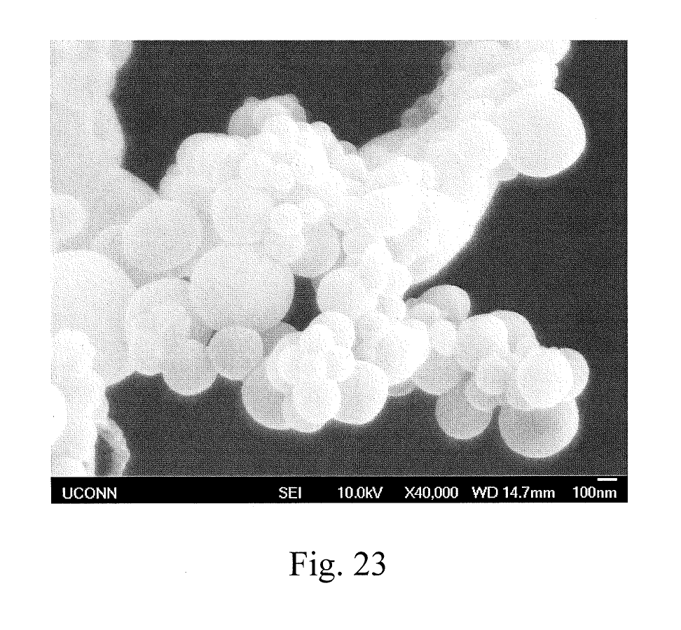

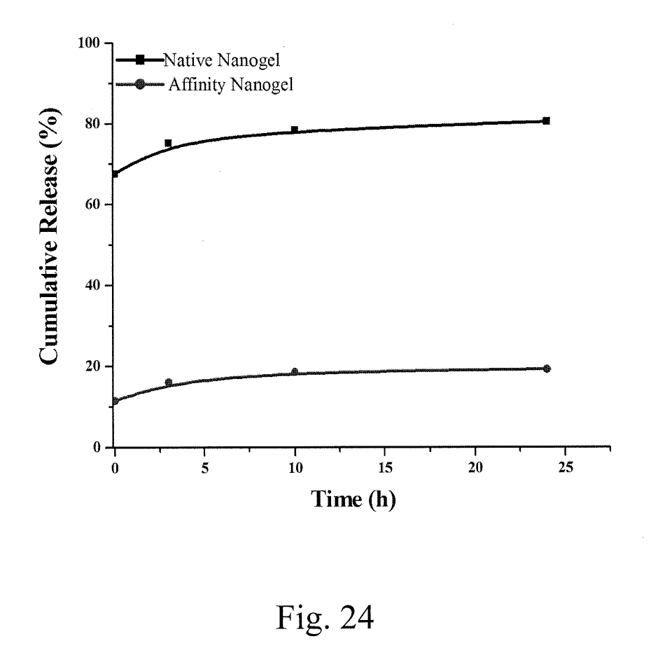

3.08 g dioctyl sodium sulfosuccinate (AOT) and 1.08 g Brij 30 were mixed with 6 mL of n-hexane. 5 nmol acrydited anti-PDGF-BB aptamer was dissolved in 200 uL PBS containing acylamide and bisacrylamide (T10%, C3.3%) and then mixed with 20 uL of 20% ammonium persulfate (APS). The mixture was added dropwise into 6 mL of n-hexane. 20 uL TEMED was add to accelerate the polymerization. After the evaporation of n-hexane, 5 mL of ethanol was added to precipitate the nanogel particles. After centrifugal, the particles were washed with ethanol for another 4 times. The nanogel particles were dried in vacuum and examined with SEM (FIG. 23). The controlled release experiment was also performed as described above. As shown in FIG. 24, release of PDGF-BB was significantly slowed in affinity nanogels as compared to native nanogels where affinity nanogel is the hydrogel functionalized with nucleic acid aptamers and native nanogel is the hydrogel without the aptamers. The results clearly demonstrated that aptamer-functionalized nano scale gel can be used to control protein release.

### Example 8

### A Method of Truncating Aptamers for Effector Discovery

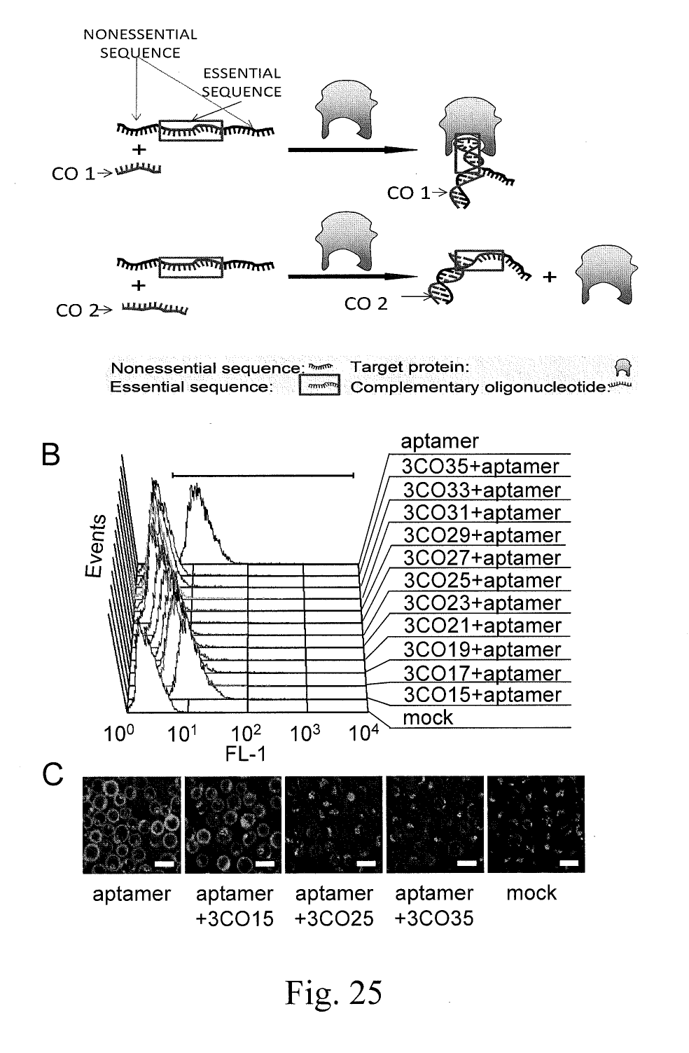

The present invention provides a novel method, in which complementary oligonucleotides are used as molecular guides to map essential or non-essential nucleotides (FIG. 25). An aptamer that can bind to human T cell lymphoblast-like CCRF-CEM cells was used as a model, since the binding of this aptamer to CCRF-CEM cells can be facilely examined with both flow cytometry and confocal microscopy imaging. Representative results of aptamer truncation are shown in FIGS. 28B and 28C. The results were acquired from both flow cytometry (BD FACSCalibur flow cytometer) and confocal microscopy (Leica SP2 confocal laser scanning microscope). These results demonstrated that this novel mapping method can be used for the discovery of truncated aptamers having only the essential sequence.

The teachings of Soontornworajit et al. “Hydrogel functionalization with DNA aptamer for sustained PDGF-BB release” Chem. Commun., (2010) 46:1857-1859; Soontornworajit et al., “Hydrogel functionalization with aptamers for sustained protein release” Chem. Commun., Supporting Information (2010) 46:S1-S7; Soontornworajit et al. “Aptamer-Functionalized in situ injectable hydrogel for controlled protein release” Biomacromolecules, (2010) 11: 2724-2730; and Soontornworajit et al. “A hybrid particle-hydrogel composite for oligonucleotide-mediated pulsatile protein release” Soft Matter (2010), 6:4255-4261 are incorporated herein by reference in their entirety.

While this invention has been particularly shown and described with references to example embodiments thereof, it will be understood by those skilled in the art that various changes in form and details may be made therein without departing from the scope of the invention encompassed by the appended claims.

1. A composition for controlled release of peptides or proteins comprising a hydrogel having a plurality of affinity sites provided by nucleic acid aptamers and peptides or proteins bound to the affinity sites.

2. The composition of claim 1, wherein the nucleic acid aptamers are selected from oligonucleotide libraries for said peptides or proteins.

3. The composition of claim 1, wherein said affinity sites comprise affinity particles, wherein said affinity particles are embedded in the hydrogel and are conjugated with said nucleic acid aptamers to anchor said nucleic acid aptamers in the hydrogel.

4. The composition of claim 3, wherein said affinity particles are microbeads.

5. The composition of claim 3, wherein said affinity particles are nanobeads.

6. The composition of claim 3, wherein said affinity particles are polystyrene particles.

7. The composition of claim 3, wherein said affinity particles are streptavidin coated particles.

8. The composition of claim 7, wherein said nucleic acid aptamers are biotinylated.

9. The composition of claim 1, wherein said nucleic acid aptamers are attached directly to said hydrogel to create the affinity sites.

10. The composition of claim 1, wherein said hydrogel is a natural or synthetic polymer.

11. The composition of claim 10, wherein said hydrogel is biocompatible.

12. The composition of claim 11, wherein said hydrogel is selected from the group consisting of polyurethane, silicone, copolymers of silicone and polyurethane, polyolefins such as polyisobutylene and polyisoprene, nitrile, neoprene, polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate), copolymers of acrylates with N-vinyl pyrrolidone, N-vinyl lactams, acrylamide, polyurethanes, polyacrylonitrile, poloxamer, agarose, methylcellulose, hyaluronan, collagen, and alginate.

13. The composition of claim 1, further comprising complementary oligonucleotides that bind to said nucleic acid aptamers and accelerate the release of said peptide or proteins from said nucleic acid aptamers.

14. The composition of claim 13, wherein said nucleic acid aptamers and/or said complementary oligonucleotides are fluorescently labeled for detection.

15. A method of controlling peptide or protein release from a hydrogel, comprising providing a hydrogel comprising a plurality of affinity sites comprising nucleic acid aptamers and peptides or proteins whose release from the hydrogel is to be controlled, wherein said peptides or proteins are bound to said nucleic acid aptamers at predetermined affinity suitable for a desired release rate, thereby controlling peptide or protein release from the hydrogel.

16. The method of claim 15, wherein said affinity sites comprise affinity particles, wherein said affinity particles are embedded in the hydrogel and said affinity particles are conjugated with said nucleic acid aptamers to anchor said nucleic acid aptamers in the hydrogel.

17. The method of claim 15, wherein said nucleic acid aptamers are attached directly to said hydrogel to create the affinity sites.

18. The method of claim 15, further comprising, accelerating aptamer-peptide or aptamer-protein dissociation by introducing complementary oligonucleotides (COs) that hybridize with said nucleic acid aptamers, wherein the hybridization of said complementary oligonucleotides with said high affinity nucleic acid aptamers causes acceleration of peptide or protein release from the hydrogel.

19. The method of claim 18, wherein said peptides or proteins are released for a defined time period and/or amount.

20. A method for making a formulation for controlled peptide or protein release comprising:
a) coating or conjugating nucleic acid aptamers on the surface of micro- or nano-particles;
b) contacting said nucleic acid aptamer-coated particles with peptides or proteins having binding affinity to said nucleic acid aptamers under conditions in which said peptides or proteins bind to said nucleic acid aptamer, wherein release of said peptides or proteins are to be controlled;
c) transferring the aptmer-coated particles saturated with said peptides or proteins into a pre-gelation polymer solution; and
d) forming a gel having a porous matrix which contains said nucleic acid aptamer-coated particles, wherein said peptides or proteins are bound to said nucleic acid aptamers.

21. A method for making a formulation for controlled peptide or protein release comprising:
a) providing nucleic acid aptamers having a functional group for conjugating said nucleic acid aptamers with a gel
b) conjugating said nucleic acid aptamers with said gel; and
c) contacting said nucleic acid aptamers with peptides or proteins under conditions that the peptides or proteins bind to said nucleic acid aptamers, wherein said peptides or proteins for which release is to be controlled have binding affinity to said nucleic acid aptamers.

22. The method according to claim 21, wherein said gel is formed under a physiological condition in which said peptides or proteins retain original function.

23. The method according to claim 21, wherein said gel is a hydrogel.

24. The method of claim 23, wherein said hydrogel is biocompatible.

25. The method of claim 23, wherein said hydrogel is a natural or synthetic polymer.

26. The method of claim 23, wherein said hydrogel is selected from the group consisting of polyurethane, silicone, copolymers of silicone, polyurethane, polyolefins such as polyisobutylene and polyisoprene, nitrile, neoprene, polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(-hydroxy ethyl methacrylate), copolymers of acrylates with N-vinyl pyrrolidone, N-vinyl lactams, acrylamide, polyurethanes, polyacrylonitrile, poloxamer, agarose, methylcellulose, hyaluronan, collagen, and alginate.

27. The method of claim 20, wherein said affinity particles are microbeads.

28. The method of claim 20, wherein said affinity particles are nanobeads.

29. The method of claim 20, wherein said affinity particles are polystyrene particles.

30. The method of claim 20, wherein said affinity particles are streptavidin coated particles.

31. The method of claim 30, wherein said one or more nucleic acid aptamers are biotinylated.

32. A method for delivering peptides or proteins to a subject in need of such peptides or proteins, comprising, administering in a physiologically effective amount to the subject in need of said peptides or proteins a formulation comprising a hydrogel having affinity sites comprising a plurality of nucleic acid aptamers having high affinity to said peptides or proteins, wherein said peptides and proteins are bound to the nucleic acid aptamers.

33. The method of claim 32, wherein the subject is a patient in need of treatment for short stature, Turner's syndrome or chronic renal failure in humans.

34. The method of claim 33, wherein the peptides and proteins are human growth hormone.

35. The method of claim 32, wherein the subject is a dairy cow, and the peptides and proteins are bovine somatotropin.

36. A kit for delivering peptides or proteins in a controlled manner comprising:
(a) a container;
(b) a formulation in said container, the formulation comprising a hydrogel having affinity sites comprising nucleic acid aptamers having high affinity to said peptides or proteins whose release is to be controlled, wherein said peptides or proteins are bound to the nucleic acid aptamers; and
(c) instructions for use.

37. A kit for delivering peptides or proteins in a controlled manner comprising:
(a) a container;
(b) a formulation in said container, the formulation comprising a hydrogel comprising micro- or nano-particles conjugated with nucleic acid aptamers having high affinity to said peptides or proteins for which release is to be controlled and having said peptides or proteins bound to the nucleic acid aptamers; and
(c) instructions for use.

38. The kit according to claim 37, further comprising complementary oligonucleotides that hybridize with said high affinity nucleic acid aptamers.

39. The kit according to claim 37, further comprising catalytic or cross-linking agent for accelerating polymerization of said hydrogel.

40. Use of a hydrogel for controlled release of peptides or proteins in the manufacture of a medicament for the treatment of a disease in a subject in need of the peptide or proteins, said hydrogel comprising affinity sites comprising nucleic acid aptamers having high affinity to said peptides or proteins and said peptides or proteins bound to the nucleic acid aptamers.

41. A composition for controlled release of peptides or proteins comprising a porous matrix having a plurality of affinity sites provided by nucleic acid aptamers and peptides or proteins bound to the affinity sites.

42. A composition for controlled release of peptides or proteins comprising a porous matrix having a plurality of affinity sites provided by nucleic acid aptamers and having peptides or proteins bound to the affinity sites.

43. A composition for controlled release of peptides or proteins comprising a hydrogel having a plurality of affinity sites provided by nucleic acid aptamers and having peptides or proteins bound to the affinity sites.

44. The composition according to claim 41, wherein said porous matrix is biocompatible.
